(19) United States
(12) Patent Application Publication
(10) Pub. No.: US 2025/0327015 A1
(43) Pub. Date: Oct. 23, 2025

(54) METHODS AND SYSTEMS FOR RESUMING CELLULAR THERAPY MANUFACTURING

(71) Applicant: Global Life Science Solutions USA, Marlborough, MA (US)

(72) Inventors: Panteleimon ZOTOS, Marlborough, MA (US); Antoine Claude Jean DAVID, Marlborough, MA (US); David BORGEAUD, Marlborough, MA (US)

(21) Appl. No.: 18/641,751

(22) Filed: Apr. 22, 2024

(51) Int. Cl. C12M 1/36; C12M 1/00; C12M 1/34; C12N 5/0783

(52) U.S. Cl. CPC C12M 41/48; C12M 23/50; C12M 41/06; C12M 41/12; C12M 41/34; C12N 5/0636

(57) ABSTRACT

A method is provided for resuming a bioprocessing system that is configured to manufacture cells for a cellular therapy. The method, performed by one or more processors and using the bioprocessing system, comprises: executing one or more first sub-state machines, of a plurality of sub-state machines for manufacturing the cells; storing, in an external system, a plurality of states; based on detecting an event, pausing the manufacturing of the cells; based on receiving user input indicating to resume the manufacturing of the cells, retrieving a saved state from the external system; comparing the saved state with the state machine to determine a sub-state machine, from the one or more first sub-state machines, that was most recently executed by the bioprocessing system prior to pausing the manufacturing of the cells; and resuming the manufacturing of the cells based on the determined sub-state machine.

### FIELD

The present disclosure relates to a system resume scheme configured to resume cellular therapy manufacturing within a bioprocessing system. This scheme is applicable to bioprocessing applications where the system resume scheme is implemented.

### BACKGROUND

Various medical therapies involve the extraction, culture, and expansion of cells for use in downstream therapeutic processes. For example, cellular therapy techniques include chimeric antigen receptor (CAR) T-cell therapy. Generally, CAR T-cell therapy redirects a patient's T cells to specifically target and destroy tumor cells. The basic principle of CAR T-cell design involves recombinant receptors that combine antigen-binding and T-cell activating functions. The general premise of CAR T-cell therapy is to artificially generate T-cells targeted to markers found on cancer cells. Scientists can remove T-cells from a person, genetically alter them, and put them back into the patient for them to attack the cancer cells. CAR T-cells may be derived from either a patient's own blood (autologous) or derived from another healthy donor (allogenic).

The first step in the production of CAR T-cells involves using apheresis, e.g., leukocyte apheresis, to remove blood from a patient's body and separate the leukocytes. After a sufficient quantity of leukocytes have been harvested, the leukapheresis product is enriched for T-cells, which involves depleting unwanted cell types. T-cell subsets having particular biomarkers can then, if desired, be isolated from the enriched sub-population using specific antibody conjugates or markers.

After isolation of targeted T-cells, the cells are activated in a certain environment in which they can actively proliferate. For example, the cells may be activated using magnetic beads coated with anti-cluster of differentiation 3 (anti-CD3)/anti-cluster of differentiation 28 (anti-CD28) monoclonal antibodies or cell-based artificial antigen presenting cells (aAPCs), which may be removed from the culture using magnetic separation. The T-cells are then transduced with CAR genes by either an integrating gammaretrovirus (RV) or by lentivirus (LV) vectors. The viral vector uses viral machinery to attach to the patient cells, and, upon entry into the cells, the vector introduces genetic material in the form of RNA. In the case of CAR-T cell therapy, this genetic material encodes the CAR. The RNA is reverse-transcribed into DNA and permanently integrates into the genome of the patient cells; allowing CAR expression to be maintained as the cells divide and are grown to large numbers in a bioreactor. The CAR is then transcribed and translated by the patient cells, and the CAR is expressed on the cell surface.

After the T cells are activated and transduced with the CAR-encoding viral vector, the cells are expanded to large numbers in a bioreactor to achieve a desired cell density. After expansion, the cells are harvested, washed, concentrated and formulated for infusion into a patient.

Existing systems and methods for manufacturing cell therapy products typically involve numerous intricate operations across an extended production cycle, spanning many days or weeks. Interruptions to the manufacturing process can potentially result in significant losses. However, existing technologies lack solutions for efficiently recovering interrupted processes.

### SUMMARY

A first aspect of the present disclosure provides a method for resuming a bioprocessing system that is configured to manufacture cells for a cellular therapy, the method comprising: executing, by one or more processors and using the bioprocessing system, one or more first sub-state machines, of a plurality of sub-state machines for manufacturing the cells, wherein each of the plurality of sub-state machines is associated with a node within a state machine, wherein the state machine comprises a decision tree with a plurality of branches and a plurality of nodes; storing, by the one or more processors and in an external system, a plurality of states, wherein each of the plurality of states is saved into the external system based on completion of a state within a first sub-state machine, from the one or more first sub-state machine, for manufacturing the cells; based on detecting an event, pausing, by the one or more processors, the manufacturing of the cells; based on receiving user input indicating to resume the manufacturing of the cells, retrieving, by the one or more processors, a saved state from the external system, wherein the saved state is a state that was saved last to the external system; comparing, by the one or more processors, the saved state with the state machine to determine a sub-state machine, from the one or more first sub-state machines, that was most recently executed by the bioprocessing system prior to pausing the manufacturing of the cells; and resuming, by the one or more processors, the manufacturing of the cells based on the determined sub-state machine.

According to an implementation of the first aspect, each of the plurality of states stored in the external system is associated with a state identifier (ID). Comparing the saved state with the state machine to determine the sub-state machine comprises: comparing the state ID of the saved state with the state machine to determine a sub-state machine, from the plurality of sub-state machines, having a state with the same state ID as the saved state; and determining the sub-state machine based on the comparison result.

According to an implementation of the first aspect, the method further comprises: storing, by the one or more processors and in the external system, a plurality of variables associated with operating conditions of the bioprocessing system, wherein a subset of the plurality of variables is saved into the external system based on completion of the sub-state machine, from the one or more first sub-state machines, for manufacturing the cells, and wherein resuming the manufacturing of the cells is further based on using the subset of the plurality of variables associated with the determined sub-state machine.

According to an implementation of the first aspect, executing the sub-state machine, from the one or more first sub-state machines, begins with default values for the subset of the plurality of variables, and wherein the subset of the plurality of variables is periodically updated during the execution of the sub-state machine from the one or more first sub-state machines.

According to an implementation of the first aspect, the method further comprises: receiving a plurality of first user inputs indicating a plurality of parameters associated with manufacturing the cells; and determining the one or more first sub-state machines based on the plurality of parameters.

According to an implementation of the first aspect, pausing the manufacturing of the cells based on detecting the event comprises: providing one or more first instructions to one or more devices within the bioprocessing system to initiate a safe mode. Resuming the manufacturing of the cells comprises: providing, based on the determined sub-state machine, one or more second instructions to the one or more devices within the bioprocessing system to stop the safe mode.

According to an implementation of the first aspect, the bioprocessing system maintains certain conditions to preserve the cells within the bioprocessing system in the safe mode, and providing the one or more first instructions to the one or more devices within the bioprocessing system to initiate the safe mode comprises one or more of: maintaining temperature and carbon dioxide (CO2) levels within the bioprocessing system; closing one or more pinch valves (PVs); stopping one or more pumps; restoring a tilted platform within the bioprocessing system to a horizontal position; engaging a disposable kit (DK); activating a red light; or opening one or more interlocks

According to an implementation of the first aspect, resuming the manufacturing of the cells comprises: determining a plurality of recovery processes based on the determined sub-state machine; performing the plurality of recovery processes; and after performing the plurality of recovery processes, executing a subsequent sub-state machine, from the plurality of sub-state machines, to resume the manufacturing of the cells.

According to an implementation of the first aspect, the external system is a removable storage device.

According to an implementation of the first aspect, the external system is a network attached storage (NAS), and wherein storing the plurality of states comprises providing, by the one or more processors and to the NAS, the plurality of states via a network.

According to an implementation of the first aspect, resuming the manufacturing of the cells based on the determined sub-state machine comprises: providing, to a second bioprocessing system, instructions to resume the manufacturing of the CAR T-cells based on the determined sub-state machine, wherein the second bioprocessing system is separate from the bioprocessing system that executed the one or more first sub-state machines.

According to an implementation of the first aspect, the external system comprises a database, and wherein the plurality of states are saved into the database in the external system based on a sequence of completing each sub-state machine of the plurality of sub-state machines.

According to an implementation of the first aspect, the cellular therapy is chimeric antigen receptor (CAR) T-cell therapy.

A second aspect of the present disclosure provides a non-transitory computer readable medium with instructions stored thereon for resuming a bioprocessing system that is configured to manufacture cells for a cellular therapy, wherein the instructions, when executed by one or more processors, causing the one or more processors to carry out: executing, using the bioprocessing system, one or more first sub-state machines, of a plurality of sub-state machines for manufacturing the cells, wherein each of the plurality of sub-state machine is associated with a node within a state machine, wherein the state machine comprises a decision tree with a plurality of branches and a plurality of nodes; storing, in an external system, a plurality of states, wherein each of the plurality of states is saved into the external system based on completion of a sub-state machine, from the one or more first sub-state machines, for manufacturing the cells; based on detecting an event, pausing the manufacturing of the cells; based on receiving user input indicating to resume the manufacturing of the cells, retrieving a saved state from the external system, wherein the saved state is a state that was saved last to the external system; comparing the saved state with the state machine to determine a sub-state machine, from the one or more first sub-state machines, that was most recently executed by the bioprocessing system prior to pausing the manufacturing of the cells; and resuming the manufacturing of the cells based on the determined sub-state machine.

According to an implementation of the second aspect, each of the plurality of states stored in the external system is associated with a state identifier (ID). Comparing the saved state with the state machine to determine the sub-state machine comprises: comparing an identifier associated with the saved state with the state machine to determine a sub-state machine, from the plurality of sub-state machines, having a state with the same state ID as the saved state; and determining the sub-state machine based on the comparison result.

According to an implementation of the second aspect, the instructions, when executed by one or more processors, cause the one or more processors to further carry out: storing, in the external system, a plurality of variables associated with operating conditions of the bioprocessing system, wherein a subset of the plurality of variables is saved into the external system based on completion of the sub-state machine, from the one or more first sub-state machines, for manufacturing the cells, and wherein resuming the manufacturing of the cells is further based on using the subset of the plurality of variables associated with the determined sub-state machine.

According to an implementation of the second aspect, executing the sub-state machine begins with default values for the subset of the plurality of variables, and wherein the subset of the plurality of variables is periodically updated during the execution of the sub-state machine from the one or more first sub-state machines.

According to an implementation of the second aspect, the instructions, when executed by one or more processors, cause the one or more processors to further carry out: receiving a plurality of first user inputs indicating a plurality of parameters associated with manufacturing the cells; and determining the one or more first sub-state machines based on the plurality of parameters.

According to an implementation of the second aspect, pausing the manufacturing of the cells based on detecting the event comprises: providing one or more first instructions to one or more devices within the bioprocessing system to initiate a safe mode. Resuming the manufacturing of the cells comprises: providing, based on the determined sub-state machine, one or more second instructions to the one or more devices within the bioprocessing system to stop the safe mode.

According to an implementation of the second aspect, resuming the manufacturing of the cells comprises: determining a plurality of recovery processes based on the determined sub-state machine; performing the plurality of recovery processes; and after performing the plurality of recovery processes, executing a subsequent sub-state machine, from the plurality of sub-state machines, to resume the manufacturing of the cells.

A third aspect of the present disclosure provides a system for resuming a bioprocessing system that is configured to manufacture cells for a cellular therapy. The system comprising: a first device in communication with one or more processors; and the one or more processors configured to: perform, using the bioprocessing system, one or more first sub-state machines, of a plurality of sub-state machines for manufacturing the cells, wherein each of the plurality of sub-state machines is associated with a node from a state machine, wherein the state machine comprises a decision tree with a plurality of branches and a plurality of nodes; store, in the first device, a plurality of states, wherein each of the plurality of states is saved into the first device based on completion of a sub-state machine, from the one or more first sub-state machines, for manufacturing the cells; based on detecting an event, pause the manufacturing of the cells; based on receiving user input indicating to resume the manufacturing of the cells, retrieving a saved state from the first device, wherein the saved state is a state that was saved last to the first device; comparing the saved state with the state machine to determine a sub-state machine, from the one or more first sub-state machines, that was most recently executed by the bioprocessing system prior to pausing the manufacturing of the cells; and resuming the manufacturing of the cells based on the determined sub-state machine.

All examples and features mentioned herein may be combined in any technically possible way.

### BRIEF DESCRIPTION OF THE DRAWINGS

The subject technology will be described in even greater detail below based on the exemplary figures, but is not limited to the examples. All features described and/or illustrated herein can be used alone or combined in different combinations. The features and advantages of various examples will become apparent by reading the following detailed description with reference to the attached drawings which illustrate the following:

FIG. 1A is a simplified block diagram depicting an exemplary environment in accordance with an example of the present disclosure.

FIG. 1B is a schematic illustration of an exemplary bioprocessing system in accordance with an example of the present disclosure.

FIG. 1C is a block diagram of an exemplary control system in accordance with an example of the present disclosure.

FIG. 2 is a simplified diagram depicting an exemplary state machine in accordance with one or more examples of the present disclosure.

FIG. 3 is an exemplary process for resuming a bioprocessing system in accordance with one or more examples of the present disclosure.

FIG. 4 is an exemplary workflow with an interruption event in accordance with one or more examples of the present disclosure.

FIG. 5 is a simplified block diagram depicting exemplary recovery processes in accordance with one or more examples of the present disclosure.

FIG. 6 is an exemplary workflow to resume a state machine in accordance with one or more examples of the present disclosure.

FIG. 7 is an exemplary workflow to resume a manufacturing process in accordance with one or more examples of the present disclosure.

FIG. 8A depicts an exemplary environment in accordance with an example of the present disclosure.

FIG. 8B is an exemplary workflow for resuming an application with reference to FIG. 8A.

FIG. 9A depicts an exemplary environment in accordance with an example of the present disclosure.

FIG. 9B is an exemplary workflow for resuming an application with reference to FIG. 9A.

It should be understood that the drawings are not necessarily to scale and that the disclosed embodiments are sometimes illustrated diagrammatically and in partial views. In certain instances, details which are not necessary for an understanding of this disclosure or which render other details difficult to perceive may have been omitted. It should be understood that this disclosure is not limited to the particular embodiments illustrated herein.

### DETAILED DESCRIPTION

Examples of the presented application will now be described more fully hereinafter with reference to the accompanying FIGs., in which some, but not all, examples of the application are shown. Indeed, the application may be exemplified in different forms and should not be construed as limited to the examples set forth herein; rather, these examples are provided so that the application will satisfy applicable legal requirements. Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.”

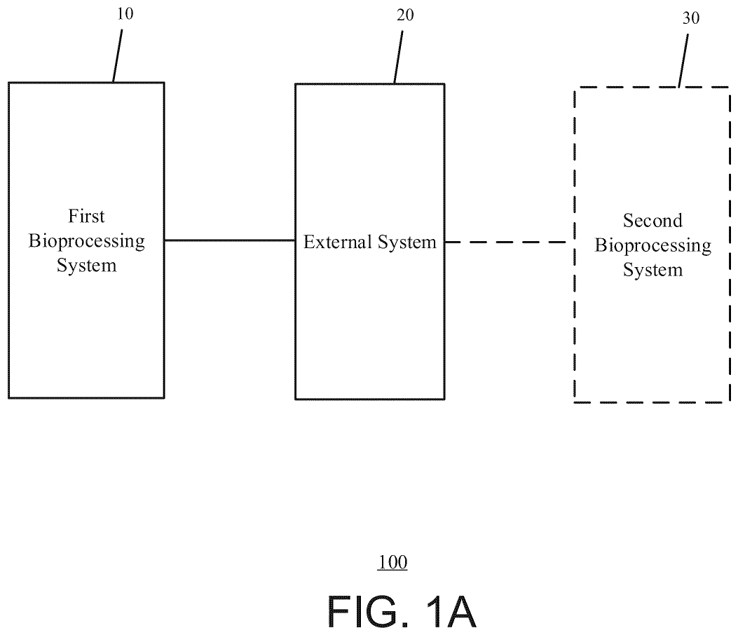

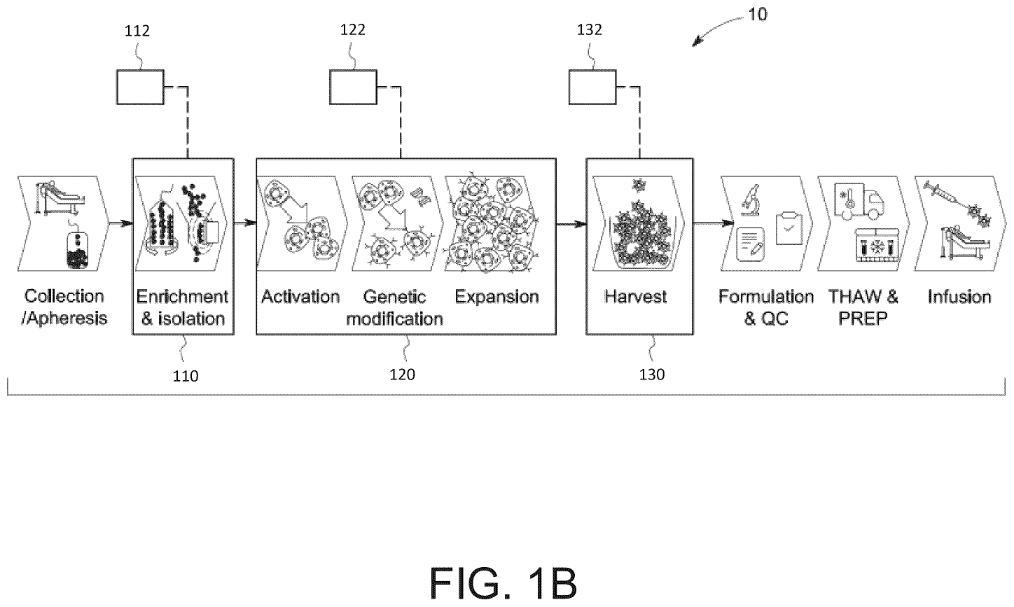

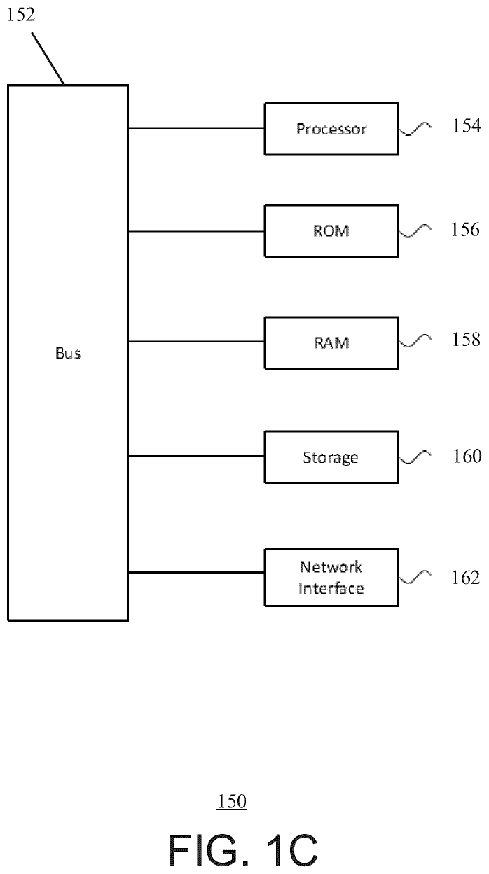

Systems, methods, and computer program products are herein disclosed that provide for resuming a bioprocessing system for the manufacturing of cells (e.g., therapeutic cells) for a cellular therapy. Cell (or cellular) therapy (CT) is a therapy that transfers autologous or allogeneic cellular materials into a patient to effectuate a medicinal effect, such as replacing or repairing damaged tissues and/or cells. FIG. 1A is a simplified block diagram depicting an exemplary environment in accordance with an example of the present disclosure. The environment 100 includes a first bioprocessing system 10, an external system 20, and an optional second bioprocessing system 30.

The first bioprocessing system 10 (bioprocessing system 10) includes various hardware and software components to carry out the manufacturing process disclosed in the present disclosure. An example of a bioprocessing system configured for use in the manufacture of cellular immunotherapies will be detailed below with reference to FIG. 1B.

The bioprocessing system 10 may be operated under the control of a control system, which may include a centralized control device and/or one or more independent control devices. The control system and/or the control device may be integrated in, connected to, and/or in communication with the bioprocessing system 10 as well as other entities (e.g., the external system 20 and/or the second bioprocessing system 30) within environment 100. The control system and/or the control device may include one or more processors, such as one or more central processing units (CPUs), controller, and/or logic, that executes computer executable instructions for performing the functions, processes, and/or methods described herein. An example of a control system will be detailed below with reference to FIG. 1C. A controller device may include some or all of the components within a control system.

Although the entities within environment 100 may be described below and/or depicted in the FIGs. as being singular entities, it will be appreciated that the entities and functionalities discussed herein may be implemented by and/or include one or more entities. For instance, the bioprocessing systems 10/30 and/or the external system 20 may include a plurality of modules, devices, systems, and/or servers that are spread across multiple different geographical locations and communicate with each other using direct connections and/or a suitable network.

The external system 20 is in communication with to the bioprocessing system 10 and configured to collaborate with the bioprocessing system 10 in order to restore suitable information for resuming the bioprocessing system 10 from an event of interruption.

Additionally and/or alternatively, the external system 20 may be in communication with to the second bioprocessing system 30 and configured to collaborate with the second bioprocessing system 30 in order to resume the interrupted process on the bioprocessing system 10. For instance, the external system 20 may resume the interrupted process on the second bioprocessing system 30.

In some examples, the control system may instruct the bioprocessing system 10 to continuously or periodically save, store, upload, modify, and/or update a set of variable values and/or a set of parameters in the external system 10 corresponding to a procedure during its operation. Furthermore, upon the completion of a procedure, the control system may instruct the bioprocessing system 10 to store the relevant state values in the external system 20 to record the completed procedure. In the event of an interruption, the control system may retrieve the stored state information from the external system 20 to determine the current state of the bioprocessing system 10, the previous completed state of the bioprocessing system 10, and/or the steps/operations to resume the manufacturing process on the bioprocessing system 10 or another bioprocessing system 30.

In some examples, the external system 20 may be any type of removable data storage system that allows users to insert or remove storage medium from the bioprocessing system 10 or any other processing system connected to the bioprocessing system 10. Examples of removable storage systems include but not limited to universal serial bus (USB) flash drives, external hard drivers, secure digital (SD) cards, MicroSD cards, optical discs (e.g., CDs, DVDs, or Blu-ray Discs), portable solid-state drives (SSDs), etc.

In some instances, the external system 20 may include a network attached storage (NAS). NAS is a dedicated file storage device or server that is connected to a network, providing centralized data storage and access to multiple users and heterogeneous clients. The external system 20 may be part of a NAS system designed to serve as a shared storage resource accessible over a network of any suitable type.

In some variations, the entities within the environment 100 such as the bioprocessing system 10, the external system 20, and/or the bioprocessing system 30 may be in communication with other devices and/or systems within the environment 100 via a network. The network may be a global area network (GAN) such as the Internet, a wide area network (WAN), a local area network (LAN), or any other type of network or combination of networks. The network may provide a wireline, wireless, or a combination of wireline and wireless communication between the entities within the environment 100.

It will be appreciated that the exemplary environment depicted in FIG. 1A is merely an example, and that the principles discussed herein may also be applicable to other situations-for example, including other types of devices, systems, and network configurations. For example, the functionalities of the bioprocessing system 10/30 and/or the external system 20 may be separated into multiple different entities.

FIG. 1B is a schematic illustration of an exemplary bioprocessing system in accordance with an example of the present disclosure. The bioprocessing system as shown in FIG. 1B may be embodied as the bioprocessing system 10 and/or the second bioprocessing system 30 within the environment 100 as depicted in FIG. 1A.

The bioprocessing system 10 is configured for use in the manufacture of cellular immunotherapies (e.g., autologous cellular immunotherapies), where, for example, human blood, fluid, tissue, or cell sample is collected, and a cellular therapy is generated from or based on the collected sample. One type of cellular immunotherapy that may be manufactured using the bioprocessing system 10 is chimeric antigen receptor (CAR) T-cell therapy, although other cellular therapies may also be produced using the system 10 or aspects thereof without departing from the broader aspects of the present disclosure. As illustrated in FIG. 1B, the manufacture of a CAR T-cell therapy generally begins with collection of a patient's blood and separation of the lymphocytes through apheresis. Collection/apheresis may take place in a clinical setting, and the apheresis product is then sent to a laboratory or manufacturing facility for production of CAR-T-cells. Once the apheresis product is received for processing, a desired cell population (e.g., white blood cells) is enriched for or separated from the collected blood for manufacturing the cellular therapy, and target cells of interest are isolated from the initial cell mixture. The target cells of interest are then activated, genetically modified to specifically target and destroy tumor cells, and expanded to achieve a desired cell density. After expansion, the cells are harvested, and a dose is formulated. The formulation is often then cryopreserved and delivered to a clinical setting for thawing, preparation and, finally, infusion into the patient.

With further reference to FIG. 1B, the bioprocessing system 10 includes a plurality of distinct modules (e.g., subsystems) that are each configured to carry out a particular subset of manufacturing processes in a substantially automated, functionally-closed, and scalable manner. For example, the bioprocessing system 10 includes a first module 110 configured to carry out the processes of enrichment and isolation, a second module 120 configured to carry out the processes of activation, genetic modification and expansion, and a third module 130 configured to carry out the process of harvesting the expanded cell population. In an embodiment, each module 110, 120, 130 may be communicatively coupled to a dedicated controller (e.g., first controller 112, second controller 122, and third controller 132, respectively). The controllers 112, 122 and 132 are configured to provide substantially automated control over the manufacturing processes within each module. While the first module 110, second module 120, and third module 130 are illustrated as including dedicated controllers for controlling the operation of each module, it is contemplated that a master control system (e.g., the control system and/or the control device) may be utilized to provide global control over the three modules. Each module 110, 120, 130 is configured to work in concert with the other modules to form a single, coherent bioprocessing system 10.

By automating the processes within each module, product consistency from each module can be increased and costs associated with extensive manual manipulations reduced. In addition, each module 110, 120, 130 is substantially functionally closed, which helps ensure patient safety by decreasing the risk of outside contamination, ensures regulatory compliance, and helps avoid the costs associated with open systems. Moreover, each module 110, 120, 130 is scalable, to support both development at low patient numbers and commercial manufacturing at high patient numbers.

With further reference to FIG. 1B, the particular manner in which the processes are compartmentalized in distinct modules that each provide for closed and automated bioprocessing allows for efficient utilization of capital equipment. As will be appreciated, the process of expanding the cell population to achieve a desired cell density prior to harvest and formulation is typically the most time-consuming process in the manufacturing process, while the enrichment and isolation processes, the harvesting and formulation processes, and the activation and genetic modification processes are much less time consuming. For example, the processes of enrichment, isolation, activation and genetic modification of cells can take place rather quickly, while expansion of the genetically modified cells takes place very slowly. Accordingly, manufacture of a cellular therapy from a first sample (e.g., the blood of a first patient) would progress quickly until the expansion process, which requires a substantial amount of time to achieve a desired cell density for harvest. With a fully automated system, the entire process/system would be monopolized by the expansion equipment performing expansion of the cells from the first sample, and processing of a second sample could not begin until the entire system was freed up for use. In this respect, with a fully-automated bioprocessing system, the entire system is essentially offline and unavailable for processing of a second sample until the entire cell therapy manufacturing process, from enrichment to harvest/formulation is completed on the first sample.

With the distinct modules depicted in FIG. 1B, the bioprocessing system 10 may be configured for parallel processing of more than one sample (from the same or different patients) to provide for more efficient utilization of capital resources. This advantage is a direct result of the particular manner in which the process processes are separated into the three modules 110, 120, 130, as alluded to above.

For instance, a single first module 110 and/or a single third module 130 may be utilized in conjunction with multiple second modules, e.g., second modules 120, in a bioprocessing system 10/30, to provide for parallel and asynchronous processing of multiple samples from the same or different patients. For example, a first apheresis product from a first patient may be enriched and isolated using the first module 110 to produce a first population of isolated target cells, and the first population of target cells may then be transferred to one of the second modules, e.g., module 120, for activation, genetic modification and expansion under control of controller 122. Once the first population of target cells is transferred out of the first module 110, the first module 110 is again available for use to process a second apheresis product from, for example, a second patient. A second population of target cells produced in the first module 110 from the sample taken from the second patient can then be transferred to another second module, for activation, genetic modification and expansion under control of a corresponding controller. Similarly, after the second population of target cells is transferred out of the first module 110, the first module is again available for use to process a third apheresis product from, for example, a third patient. A third target population of cells produced in the first module 110 from the sample taken from the third patient may then be transferred to another second module for activation, genetic modification and expansion under control of the corresponding controller. In this respect, expansion of, for example, CAR T-cells for a first patient may occur simultaneously with the expansion of CAR T-cells for a second patient, a third patient, etc. This approach also allows the post processing to occur asynchronously as needed. In other words, patient cells may not all grow at the same time. The cultures may reach the final density at different times, but the multiple second modules 120 are not linked, and the third module 130 may be used as needed. With the present disclosure, while samples may be processed in parallel, they do not have to be done in batches. Harvesting of the expanded populations of cells from the second modules may likewise be accomplished using a single third module 130 when each expanded populations of cells are ready for harvest.

Accordingly, by separating the processes of activation, genetic modification and expansion, which is the most time consuming, and which share certain operational requirements and/or require similar culture conditions, into a stand-alone, automated and functionally-closed module, the other system equipment that is utilized for enrichment, isolation, harvest and formulation is not tied up or offline while expansion of one population of cells is carried out. As a result, the manufacture of multiple cell therapies may be carried out simultaneously, maximizing equipment and floor space usage and increasing overall process and facility efficiency. It is envisioned that additional second modules 120 may be added to the bioprocessing system 10 to provide for the parallel processing of any number of cell populations, as desired. Accordingly, the bioprocessing system of the invention allows for plug-and-play like functionality, which enables a manufacturing facility to scale up or scale down with ease.

In some examples, the first module 110 may be any system or device capable of producing, from an apheresis product taken from a patient, a target population of enriched and isolated cells for use in a biological process, such as the manufacture of immunotherapies and regenerative medicines. The third module 130 may be any system or device capable of harvesting and/or formulating CAR T-cells or other modified cells produced by the second module 120 for infusion into a patient, for use in cellular immunotherapies or regenerative medicine. In certain embodiments, the first module 110 and the third module 130 are similarly or identically configured, such that the first module 110 may first be utilized for enrichment and isolation of cells (which are then transferred to the second module 120 for activation, transduction and expansion (and in some embodiments, harvesting)), and then also used at the end of the process for cell harvesting and/or formulation. In this respect, in some embodiments, the same equipment can be utilized for the front-end cell enrichment and isolation processes, as well as the back-end harvesting and/or formulation processes.

It will be appreciated that the exemplary bioprocessing system depicted in FIG. 1B is merely an example, and that the principles discussed herein may also be applicable to other situations-for example, including other types of devices, systems, and configurations. For example, the modules 110, 120, and 130 may be consolidated into a single module or distributed across a number of different entities.

FIG. 1C is a block diagram of an exemplary control system configured to control one or more entities (e.g., the bioprocessing system 10/30 and/or the external system 20) within the environment 100.

The control system 150 includes one or more processors 154, such as one or more CPUs, controller, and/or logic, that executes computer executable instructions for performing the functions, processes, and/or methods described herein. In some examples, the computer executable instructions are locally stored and accessed from a non-transitory computer readable medium, such as storage 160, which may be a hard drive or flash drive. Read Only Memory (ROM) 156 includes computer executable instructions for initializing the processor 154, while the random-access memory (RAM) 158 is the main memory for loading and processing instructions executed by the processor 154. The network interface 162 may connect to a wired network or cellular network and to a local area network or wide area network. The control system 150 may also include a bus 152 that connects the processor 154, ROM 156, RAM 158, storage 160, and/or the network interface 162. The components within the control system 150 may use the bus 152 to communicate with each other. The components within the control system 150 are merely exemplary and might not be inclusive of every component, server, device, computing platform, and/or computing apparatus within the control system 150. Additionally, and/or alternatively, the control system 150 may further include components that might not be included within every entity of environment 100.

As previously mentioned, with reference to FIG. 1B, the bioprocessing system 10 (e.g., each module or subsystem within it) is configured to carry out a number of manufacturing processes in a substantially automated manner. Each process is associated with a preconfigured set of instructions that the processer may execute to control the bioprocessing system 10 in carrying out the corresponding process. In some examples, each process may consist of a number of steps, with each step referred to as a sub-process and associated with a subset of instructions. Each process or sub-process may be defined based on initial conditions, variables during the operation, and/or termination criteria for a particular process or sub-process. Instructions for the processes and/or sub-processes may be generated automatically, semi-automatically, or fully manually, based on user inputs, default values, and/or historical data. Hereinafter, an application refers to an entire set of instructions associated with manufacturing of cells for a cellular therapy.

Furthermore, the processes or sub-processes are configured to run in a specific sequence. The sequence of running the processes or sub-processes may be represented in any suitable form. For example, a decision tree and/or a finite state machine may be utilized to control the execution of the application according to a specific sequence, in which predefined connections between nodes may indicate the execution order among the processes/sub-processes within the application. Hereinafter, the processes and sub-processes are both referred to as “processes” for brevity.

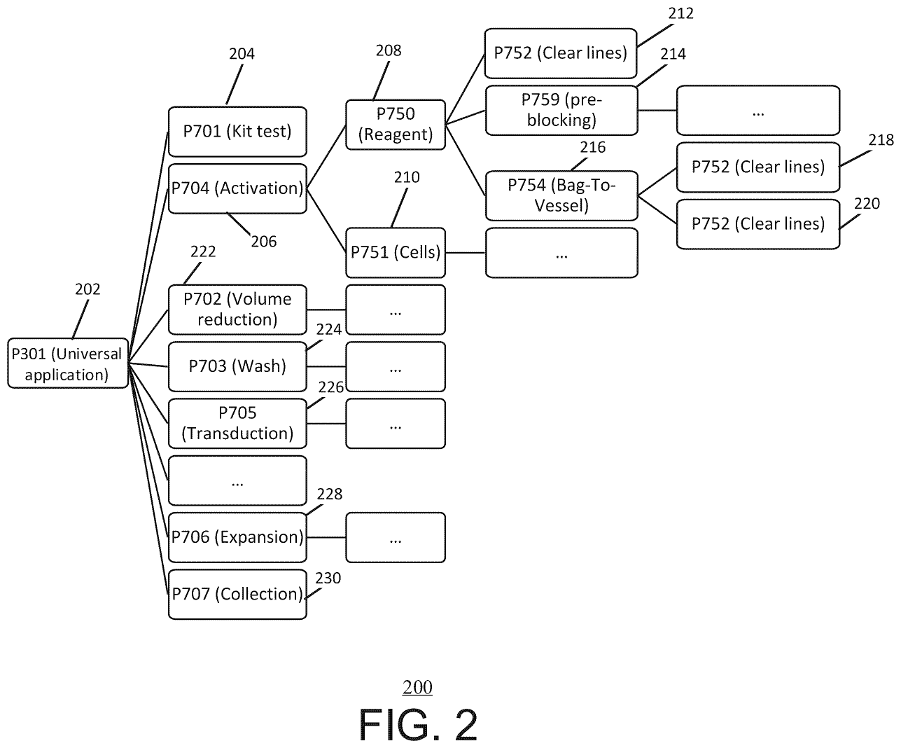

FIG. 2 is a simplified diagram depicting an exemplary state machine in accordance with one or more examples of the present disclosure. As shown in FIG. 2, the state machine 200 includes a number of connected blocks (e.g., blocks 202, 204, 206, . . . ). Each block represents a sub-state machine, which may be linked to a protocol identification (ID), such as P701 for block 204. Each sub-state machine includes a list of states that an application may transition through by executing processes/sub-processes defined in the particular application. The connections between the blocks indicate potential transitions between the corresponding blocks in a suitable direction. For instance, in one example, the state machine 200 may transition from block 202 to block 204. In another example, the state machine 200 may first move from block 208 to block 212 and then return from block 212 to block 208, enabling the execution of block 214 subsequently.

Each state within the state machine/sub-state machine is associated with an entry condition and an exit condition, contingent upon the input values. For example, with a first set of input values, the application may enter a first state, whereas with a second set of input values, the application may exit the first state and/or enter a second state. This way, the application may transition between states within a particular sub-state machine, or transition between sub-state machines within the state machine 200. The state machine may follow varied paths based on the application inputs, such as parameters, retrieved values, and/or other relevant factors. Change in the application inputs may be associated with one or more processes defined within the application. For example, the performance of a process may begin with an initial condition (e.g., aligning with the entry condition of a specific state). The changes occurring during the process may be manifested as the transformation of one or more variables. Completion of the process may be determined by a conclusion condition (e.g., corresponding to the exit condition of the specific state). Accordingly, during the course of carrying out one or more processes, the application may obtain varied inputs based on the current status of the manufacturing system (e.g., the bioprocessing system 10). Based on these inputs, the application (e.g., the state machine 200) may determine whether to move to the next state or the next sub-state machine. In other words, the performance of one or more processes by the bioprocessing system 10 may result in changes in its status, causing the application (e.g., the state machine 200) to transition through a series of states as prescribed.

A control system (e.g., one or more processors within the system) may execute the state machine 200 to control the bioprocessing system 10 (as shown in FIG. 1A or 1B) to carry out the manufacturing processes. Information pertaining to the execution of the state machine 200 may be stored in various types of data structure. For example, the control system may store relevant information associated with a sequence of executed sub-state machines and states in a stack data structure, such as a call stack. A call stack may include a list of entries, with each entry containing identification information indicating an executed state within the state machine 200. The identification information may encompass a first identification (ID), such as a protocol ID (e.g., P701 in block 204), associated with a particular sub-state machine (e.g., block 204), as well as a second ID, such as a state ID, indicating a particular state within the particular sub-state machine. Put differently, the identification information from a specific entry may be utilized to pinpoint a particular state of a particular sub-state machine within the state machine 200. The control system may append a new entry associated with a state in a sub-state machine when that particular state is executed or has recently been completed. This way, the control system may store a list of entries in the call stack corresponding to sequentially executed states in the corresponding sub-state machine(s). As such, by inspecting the list of entries stored in the call stack, the state machine 200 is capable of traversing the executed sub-state machines and relevant states in accordance with the preceding execution order. In other words, the previous execution chain may be “replayed” with guidance from the call stack.

In some instances, the control system may dynamically update a call stack during the execution of the state machine 200, for example, by performing addition, deletion, or modification to one or more entries in the call stack. For example, the addition may be facilitated by appending a new entry to the call stack as discussed above. The deletion of one or more entries may be associated with a movement from a child node (e.g., block 212) to a parent node (e.g., block 208) in the state machine 200. For example, when the state machine 200 returns from block 212 to block 208, all entries corresponding to the protocol ID 752 (in block 212) may be removed. Put another way, the sub-state machine that has been completed and will not be executed or passed through again may not be on the monitoring list (e.g., the dynamic call stack), as a resumption of the application does not need to pass through that particular sub-state machine. Therefore, the control system may focus on a series of mutually dependent sub-state machines that impact the resumption to a specific state (e.g., the crashed state) in a specific sub-state machine. In some variations, the list of entries in the call stack may maintain only one protocol ID and a corresponding state ID for a particular block in the state machine 200. For instance, when a sub-state machine in block 204 is executed, it may pass through a series of states. The control system may overwrite the state ID corresponding to the protocol ID in the call stack, according to the current state of the particular sub-state machine. To this end, in the call stack, each protocol ID for a particular block within the state machine 200 may be associated with a last-saved state ID, indicating the last executed state (e.g., completed, paused, or under other scenarios) in the corresponding sub-state machine. Additionally, when the state machine 200 returns from a child node to a parent node, the last-saved state ID corresponding to the sub-state machine in the parent node may be updated. This may involve appending a new entry or overwriting an existing entry, which may be associated with one or more additional processes (e.g., calculations, measurements) that take place. Alternatively, the state machine 200 may pass through a sub-state machine without changes to its last-saved state ID.

In some variations, the control system may store one or more call stacks during the manufacturing of cellular immunotherapies (e.g., the manufacturing of CAR T-cells). One call stack may be dynamically updated for an “open” execution chain, which refers to a series of sub-state machines that have not reached an end node (e.g., block 218). Each of the other call stacks may indicate a complete execution chain from the root node (e.g., block 202) to an end node (e.g., 212). This way, the other call stacks may be utilized as historical data for later evaluation/debugging of the application based on the outcomes from the manufacturing products.

Referring back to the state machine 200 as depicted in FIG. 2, the following describes examples of executing the state machine 200 and storing/updating a corresponding call stack. Each block in the state machine 200 corresponds to a sub-state machine with a corresponding protocol ID, such as P301 for block 202, P701 for block 204, P704 for block 206, etc. As indicated in the corresponding blocks in FIG. 2, each sub-state machine is associated with one or more manufacturing processes, such as “Universal application” (e.g., this may be the commercial name of the application) for P301, “Kit test” for P701, “Activation” for P704, etc. In some examples, each manufacturing process may include one or more sub-processes, which may be associated with transitions of states within a particular sub-state machine.

In one example, the state machine 200 may execute in accordance with the following execution chain: P301—Universal application→P704—Activation→750—Reagent (in block 208)→754—Bag-To-Vessel (in block 216)→P752—Clear lines (in block 218). During the execution, the control system may store a protocol ID P301 and a state ID 1000 to the call stack once the sub-state machine corresponding to the protocol ID P301 has been completed. The state ID 1000 is the last-saved state ID for the protocol ID P301. Then, the state machine 200 transition to the next sub-state machine with a protocol ID P704. Similarly, the control system may store the protocol ID P704 and a state ID 2000 to the call stack once the sub-state machine corresponding to the protocol ID P704 has been completed. The state ID 2000 is the last-saved state ID for the protocol ID P704. To this end, if the execution chain is completed normally, the control system may store a call stack recording the executed sub-state machines (by their protocol IDs) and the corresponding last-saved state IDs. However, if an interruption occurs in a particular state in the sub-state machine with a protocol ID P754, the state-machine 200 may be stopped at that particular state. Accordingly, the control system may store the following call stack: P301: State 1000, P704, State 2000, P750, State 3000, P754, State 1100. As indicated by the call stack, the execution of the state machine 200 was stopped at the state 1100 in the sub-state machine P754. In some instances, the control system may store intermediate states within each sub-state machine into the call stack during the execution. In some variations, the control system may overwrite the state ID within each sub-state machine during the execution, so that the call stack only stores one protocol ID with a corresponding last-saved state ID for the particular sub-state machine.

The transition from one state to a next state within a sub-state machine or transition from one sub-state machine to a next sub-state machine depends on various factors, such as the process/sub-process to be executed and/or certain criteria, parameters, and/or other factors for the execution of the process/sub-process. In some instances, the manufacturing of cellular immunotherapies (e.g., manufacturing of CAR T-cells) may be different for different drug pipelines. For example, different drug pipelines may lead to varying configurations in the bioprocessing system 10 and/or the application (e.g., as demonstrated by the state machine 200 in FIG. 2), facilitated, for example, by configuring suitable user parameters related to one or more sub-state machines in the state machine 200. As such, depending on certain criteria, parameters, and/or other factors, the control system may be configured to perform the state machine 200 with a first configuration for a first drug pipeline and the state machine 200 with a second configuration for a second drug pipeline. For example, referring to the example state machine 200 as depicted in FIG. 2, for the first and second drug pipelines, the control system may control the bioprocessing system 10 to execute the sub-state machine in block 208 (e.g., P750—Reagent). Then, based on one or more factors, the control system may control the bioprocessing system 10 to execute the sub-state machine in block 214 (e.g., P759—Pre-Blocking) for the first drug pipeline and may control the bioprocessing system 10 to execute the sub-state machine in block 216 (e.g., P754—Bag-To-Vessel) for the second drug pipeline. As such, the control system may execute the state machine 200 in various sequences, and/or execute different subsets of sub-state machines for different usage scenarios.

As detailed above, based on the protocol IDs and state IDs stored in a call stack, the control system may be able to determine the exact sub-state machines (and/or states in the sub-state machines) that were previously executed. Using the call stack as well as other suitable information (e.g., the variables, parameters, and/or other factors), the control system may resume the operation of manufacturing of the cellular immunotherapies from an interruption. This will be described in further detail below.

While executing each respective sub-state machine, the control system may monitor relevant state information during the runtime. In some instances, the control system may instruct an external system (e.g., the external system 20 in FIG. 1A) to periodically store information such as one or more variables and/or parameters. Parameters may be related to information provided by end user prior to or during an application run. The control system may determine whether to execute some or all of the processes/sub-processes defined in the particular application. As such, these parameters may influence the subset and sequence of states that the application actually traverses. The parameters may include, but are not limited to, target results (e.g., a target volume of a destination liquid bag) and/or threshold values (e.g., an alarm threshold), serving as conditions for determining completion or termination at a respective state node. The parameters may include fixed values and/or be modifiable by the end user during the run. Variables may be associated with the execution of a particular process or sub-process, evolving over time. For example, the values of the variables may indicate the progress of a bioprocessing system 10 while executing the particular process or sub-process. The variables may include, but are not limited to, sensor values, calculation results, a target report output path, a run ID, duration information, and more.

Additionally and/or alternatively, upon completion of a sub-state machine, the control system may instruct the external system to store unique state information (e.g., protocol ID, state ID, etc.) for the completed sub-state machine, the sub-state machine that is next to be performed, and/or the sub-state machine that is currently being performed. For example, the control system may store a protocol ID and a corresponding state ID associated with a respective sub-state machine. As the control system executes the sub-state machines according to the state machine 200, it may store a series of sub-state machines and corresponding states corresponding to the sequence of executing the sub-state machines. The control system may retrieve the stored information from the external system and traverse through the sub-state machines and corresponding states based on the connections between them.

For instance, after completing a sub-state machine (e.g., in block 208), the control system may instruct the external system 20 to store a protocol ID and a state ID associated with the sub-state machine 208. For example, the control system may provide instructions to the bioprocessing system 10 to store the protocol ID and corresponding state ID associated with completed sub-state machine in the external system 20. Additionally, and/or alternatively, the bioprocessing system 10 may store the protocol ID and corresponding state ID for the completed sub-state machine in the external system 20 without receiving any instructions from the control system. In some variations, the storage of the protocol ID and corresponding state ID associated with the sub-state machine may be performed after completion of the sub-state machine. Additionally, and/or alternatively, the control system may store the protocol ID and corresponding state ID(s) associated with the sub-state machine that is currently being performed and/or the sub-state machine that is next to be performed into the external system 20. In other words, the control system may store a protocol ID and a corresponding state ID for a current operating state of the bioprocessing system 10 associated with any sub-state machine and/or states defined in the state machine 200, thereby enabling the control system to determine the location within the state machine 200 to resume the manufacturing process in the event an interruption event occurs.

When an interruption event occurs, the bioprocessing system 10 may be paused at the current status or may be set to a specific mode (e.g., a safe mode) to preserve the samples (e.g., the cell samples) in the system 10. The last state of the bioprocessing system 10 may be stored in the external system 20 as the last-saved state corresponding to the specific manufacturing process. For example, the last-saved state in the external system 20 may be the most recently completed state associated with the most recently completed sub-state machine in the state machine 200. For instance, after completing the sub-state machine in block 208 (e.g., corresponding to a process “Reagent”), the control system may direct the bioprocessing system to save the state ID associated with the sub-state machine 208 into the external system 20. If an interruption event occurs prior to saving another state ID in the external system 20, the state ID associated with the completed sub-state machine may be the last saved state. Additionally, and/or alternatively, as mentioned above, the last-saved state may be another state associated with the current state such as the next state to be performed by the bioprocessing system 10. As will be described below, the control system may use the last-saved state to resume the manufacturing process.

As depicted in FIG. 2, certain blocks, such as blocks 212, 218, and 220, are associated with the same sub-state machine “Clear lines” with a protocol ID P752. In this scenario, blocks 212, 218, and 220 may correspond to the same code. As such, during the execution of blocks 212, 218, and 220, the control system may call the corresponding code at different instances.

As shown, the state machine 200 includes a plurality of sub-state machines such as sub-state machines 202-230. The state machine 200, along with its sub-state machines, are merely exemplary. In addition, certain sub-state machines are shown as “. . . ” to show that there may be further branches and/or nodes that are associated with them. These additional branches and/or nodes are not shown merely for clarity and brevity.

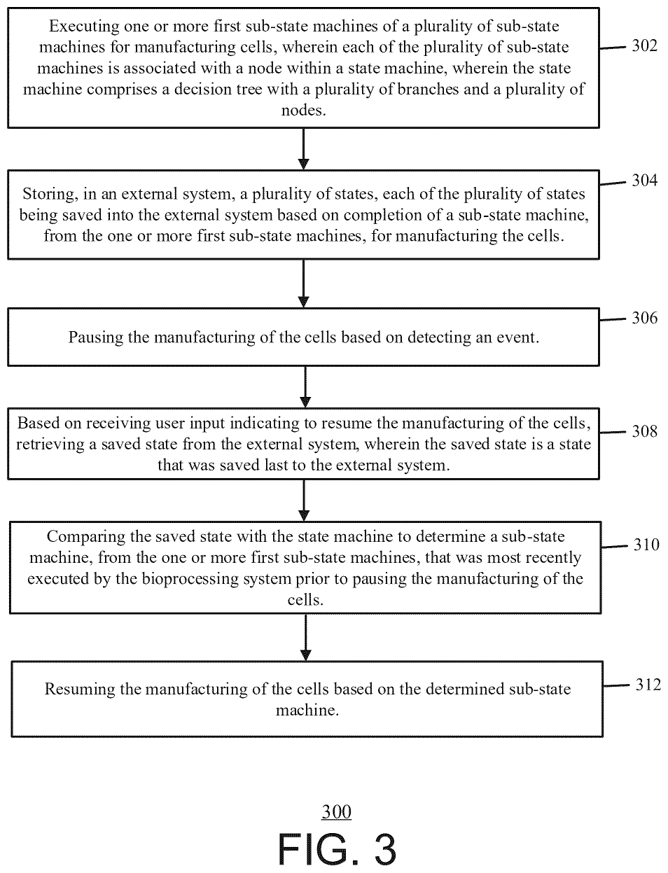

FIG. 3 is an exemplary process for resuming a bioprocessing system (e.g., bioprocessing system 10) in accordance with one or more examples of the present disclosure. The process 300 may be performed by a control system (e.g., the control system shown in FIG. 1C) configured to control the operation of the bioprocessing system 10, the second bioprocessing system 30, and/or the external system 20 within the environment 100 shown in FIG. 1A. In this example, the bioprocessing system 10 may be configured for use in the manufacture as shown in FIG. 1B for illustrative purposes. The control system may execute the state machine 200 depicted in FIG. 2 to control the operation of the bioprocessing system 10/30 and/or the external system 20. It will be recognized that any of the following blocks may be performed in any suitable order, and that the process 300 may be performed in any suitable environment. The descriptions, illustrations, and processes of FIG. 3 are merely exemplary and the process 300 may use other descriptions, illustrations, and processes for resuming a bioprocessing system for a particular manufacturing process.

At block 302, the control system, using the bioprocessing system 10, executes one or more first sub-state machines of a plurality of sub-state machines for manufacturing cells (e.g., CAR T-cells) for a cellular therapy. Each of the plurality of sub-state machines is associated with a node within a state machine 200. The state machine 200 includes a decision tree with a plurality of branches and a plurality of nodes. The sub-state machines refer to a series of nodes sequentially connected along one or more paths in the decision tree of the state machine 200. The one or more first sub-state machines refer to executed and/or completed sub-state machines.

The control system may determine the plurality of sub-state machines for the manufacturing based on user inputs. For example, the control system may receive a plurality of first user inputs indicating a plurality of parameters associated with the manufacturing and determine the plurality of sub-state machines based on the plurality of parameters. The control system may then execute the determined sub-state machines such as the first sub-state machines. For example, one or more parameters may be associated with information provided by an end user before or during the application run. The parameter(s) may indicate an order to execute (or not execute) a part of the process (or the sub-state machine), information about a target result (e.g., a volume of a destination liquid bag), an alarm threshold, or other information. In some examples, a parameter refers to a “fixed” value which may be modified during the run by end user. The execution of sub-state machines may involve changing values in one or more variables. A variable refers to data used by a corresponding process that may evolve over time. The variables may include sensor values, calculation results, output paths for target report, run IDs, duration information, and more.

At block 304, the control system stores a plurality of states in the external system 20. Each of the plurality of states is stored into the external system 20 based on completion of a state within a first sub-state machine from the state machine 200. The completion of the particular state is associated with the execution of a corresponding sub-state machine, from the one or more first sub-state machine, for manufacturing the cells. Each completed state may be associated with a unique state identifier (ID) within a particular sub-state machine. The plurality of states may be stored into the database and/or memory of the external system 20 based on completing each sub-state machine (or a particular state therein) of the plurality of sub-state machines.

For example, referring to FIG. 2, the control system may control the bioprocessing system to execute a series of sub-state machines to cause the state machine 200 to pass through a sequence of nodes in the following order: P301 (e.g., node 202)→P704 (e.g., node 206)→P750 (e.g., node 208)→P754 (e.g., node 216), and so on. During the operation, the control system may store the states as: state ID “1000” for node 202 with protocol ID P301; state ID “2000” for node 206 with protocol ID P704; state ID “3000” for node 208 with protocol ID P750; state ID “1100” for node 216 with protocol ID P754. During the execution of the sub-state machines, the bioprocessing system 10 may encounter an interruption event. In the example above, the last-saved state may be state ID “1100”, associated with the node 216 with protocol ID 754.

In some instances, the control system may store a plurality of variables associated with operating conditions of the bioprocessing system 10. The control system may store a subset of the plurality of variables into the external system based on completion of a sub-state machine, from the one or more first sub-state machines. The subset of the plurality of variables may be associated with the unique state ID (e.g., a last-saved state ID) and/or the protocol ID corresponding to the respective sub-state machine. As such, the control system may retrieve the corresponding set of variables along with the respective state ID and/or the protocol ID.

When the control system executes a respective sub-state machine, the control system may initiate the respective sub-state machine with default values for the corresponding subset of the plurality of variables. The subset of the plurality of variables may be periodically updated during the execution of the respective sub-state machine.

At block 306, the control system pauses the manufacturing of the cells based on detecting an event (e.g., an interruption event). The event may include, but is not limited to sensor failure detection, overcurrent detection, power outage, user-initiated termination, and more. The control system may instruct the bioprocessing system 10 to preserve the current status and/or enter a safe mode. For example, as mentioned above, the manufacturing of cells for cell therapy may take a substantial amount of time. Furthermore, the individuals (e.g., patients) undergoing cell therapy may have certain ailments. For instance, CAR T-cell therapy may be used to treat a patient's cancer diagnosis. Thus, a delay or restarting the manufacturing of cells for cell therapy might not be practical nor achievable. As such, the control system may use process 300 to pause and resume the manufacturing of cells to ensure that the cells are not lost due to an event. For example, at block 306, the control system may pause the manufacturing of cells to preserve the current status of cells. Additionally, and/or alternatively, while undergoing one or more processes, the control system might not be able to completely pause the process without harming the cells or cellular structure. As such, the control system may set the bioprocessing system 10 into a safe mode to preserve the condition of the cells.

In some variations, the control system may detect the event based on notifications (e.g., alerts) received from the bioprocessing system 10 (e.g., via user input) and/or by identifying abnormal status in the bioprocessing system 10.

In some examples, the control system may provide one or more first instructions to one or more devices/modules within the bioprocessing system to initiate a safe mode. The safe mode is described in further detail below.

At block 308, the control system receives user input indicating to resume the manufacturing of the cells and retrieves a saved state from the external system 20. The saved state may be the last-saved state (e.g., the most recently saved state) corresponding to the manufacturing process in the external system 20. For instance, as mentioned previously, the external system 20 may store a plurality of states associated with the sub-state machines and/or sub-routines that have been performed by the control system/the bioprocessing system 10. For example, the external system 20 may have stored the following states: state ID “1000” for the sub-state machine 202 with protocol ID P301; state ID “2000” for the sub-state machine 206 with protocol ID P704; state ID “3000” for the sub-state machine 208 with protocol ID P750; state ID “1100” for the sub-state machine 216 with protocol ID P754. The control system may determine the saved state that was last saved to the external system 20 (e.g., the saved state with a metadata indicating a time stamp that is closest to the current time). As mentioned previously, the last-saved state may be the state ID associated with the sub-state machine within the state machine 200 that was most recently completed by the bioprocessing system 10. Additionally, and/or alternatively, the last-saved state may be the currently performed state and/or the next state to be performed.

For example, in some instances, the control system may notify the user (e.g., an operator) of a resumable state machine/sub-state machine associated with the manufacturing process based on a retrieved call stack, for example, displayed on a graphic user interface on a display associated with the control system. Once the user selects to resume the state machine/sub-state machine, the control system may proceed to perform the resumption process.

In some examples, the control system may retrieve all or part of the saved states in the call stack from the external system 20. The control system may traverse the retrieved states based on the connections between the states as indicated by the decision tree of the state machine 200 or a corresponding sub-state machine.

In some instances, the control system may store the plurality of states (e.g., to perform block 304) in another suitable storage device/system and subsequently retrieve the states therefrom (e.g., to perform block 308) in various suitable usage scenarios. For example, the storage device/system may be integrated into or in communication with the control system/the bioprocessing system 10 (or the bioprocessing system 30).

At block 310, the control system compares the saved state with the state machine to determine a sub-state machine, from the one or more first sub-state machines, that was most recently executed by the bioprocessing system 10 prior to pausing the manufacturing of the cells. For example, the control system may compare the retrieved saved state from block 308 (e.g., state ID “1100” for the sub-state machine 216 with protocol ID P754) with the state machine (e.g., state machine 200) to determine the most recently performed state in the respective sub-state machine. For instance, as mentioned previously, a call stack may be stored with a plurality of unique state IDs for the corresponding sub-state machine(s). The control system may compare the retrieved saved state (e.g., with the unique state ID and/or corresponding protocol ID) from the call stack with the state machine 200 to determine the most recently executed sub-state machines. For example, based on retrieving the state ID “1100” and its corresponding protocol ID, the control system may determine that the most recently executed state is in the sub-state machine P754—Bag-To-Vessel in node 216. Additionally, and/or alternatively, the retrieved state ID “1100” may indicate the next state to be performed, as such the control system may determine that the most recently executed state is in the sub-state machine P750—Reagent in node 208.

In other words, in some examples, based on the last-saved state, the control system may determine the interrupted sub-state machine and/or the interrupted state therein, and may determine how to resume the bioprocessing system 10 based on the determination. For example, the control system may determine the necessary steps/processes to resume a bioprocessing system 10/30 from the determined last-state of the bioprocessing system 10, such as by providing instructions to the bioprocessing systems 10 or 30 to reach certain conditions, variables, and/or parameters so as to be able to resume the previously interrupted sub-state machine.

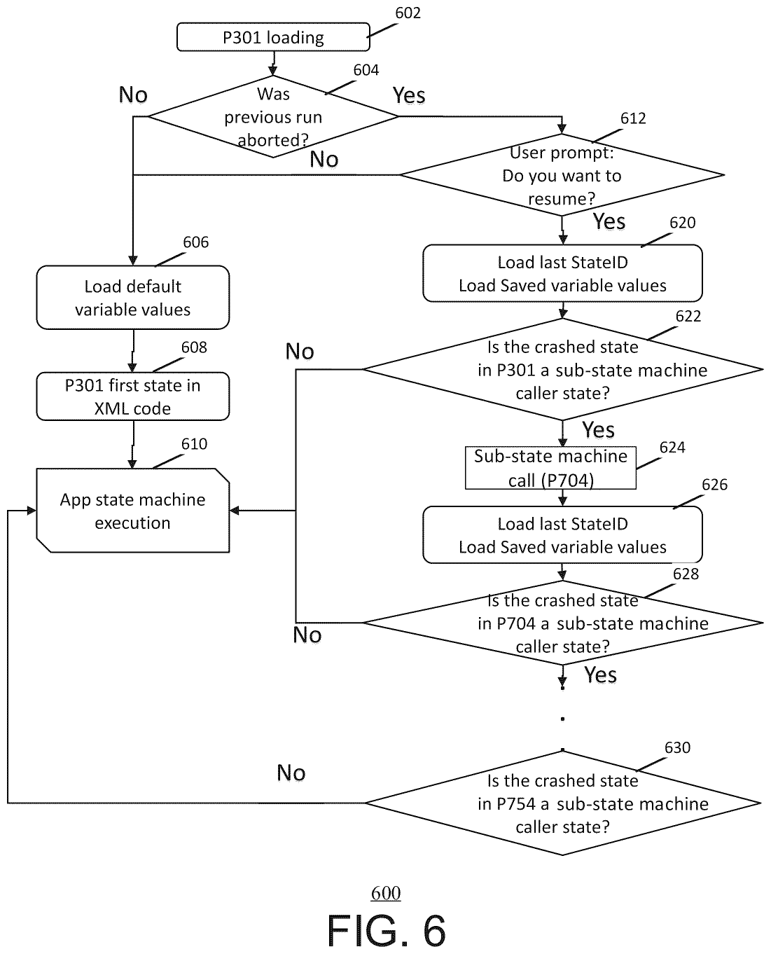

In some examples, the control system may determine the one or more first sub-state machines according to the previous execution chain and then systematically examine the status information (e.g., the state ID) associated with each sub-state machine sequentially to determine whether the corresponding sub-state machine was completed normally or interrupted (this will be elaborated with reference to FIG. 6).

At block 312, the control system resumes the manufacturing of the cells based on the determined sub-state machine. For example, the control system may resume normal state machine execution, starting from the interrupted state within the determined sub-state machine. In some variations, the control system may resume the manufacturing of the cells based on using the subset of the plurality of variables associated with the determined sub-state machine. For example, the control system may provide instructions to set the bioprocessing system 10 or 30 to certain conditions based on the subset of variables. Afterwards, the control system may resume the manufacturing of the cells.

In some examples, the control system may determine a plurality of recovery processes based on the determined sub-state machine and control the bioprocessing system 10/30 to perform the plurality of recovery processes. Subsequently, the control system may execute a subsequent sub-state machine, from the plurality of sub-state machines, to resume the manufacturing of the cells.

In further examples, the bioprocessing system 10 may enter a safe mode when interrupted, unless there in a power failure, rendering the safe mode unachievable. When the bioprocessing system 10 enters the safe mode, the control system may provide, based on the determined sub-state machine, one or more second instructions to the one or more devices/modules within the bioprocessing system 10 to stop the safe mode. The control system may then resume the manufacturing of the cells after the bioprocessing system 10 exits the safe mode.

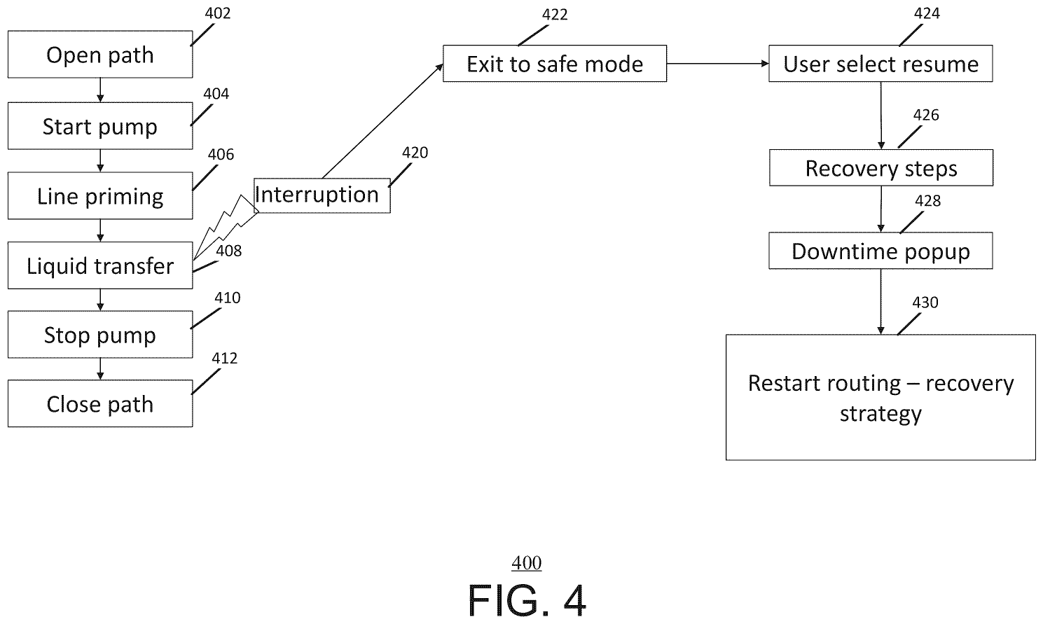

FIG. 4 is an exemplary workflow with an interruption event in accordance with one or more examples of the present disclosure. A control system controls the bioprocessing system 10/30 and the external system 20, as illustrated in FIGS. 1A-1C, to carry out the workflow 400. The exemplary blocks 402-412 may constitute a sequence of connected sub-state machines in the state machine 200, as depicted in FIG. 2. The control system may perform the process 300 illustrated in FIG. 3 to carry out the workflow 400. The descriptions, illustrations, and processes of FIG. 4 are merely exemplary and the workflow 400 may use other descriptions, illustrations, and processes for resuming a bioprocessing system from an interruption event.

As shown in the example of FIG. 4, the control system is configured to control the bioprocessing system 10 to sequentially execute blocks 402-412 for a manufacturing process according to the workflow 400.

In the course of operation, the bioprocessing system 10 (e.g., the control system thereof) successfully carries out the sub-state machines of “open path” (in block 402), “start pump” (in block 404), and “line priming” (in block 406). However, an interruption 420 takes place when the bioprocessing system 10 is engaged in the sub-state machine “liquid transfer” (in block 408). Consequently, the bioprocessing system 10 exits the sub-state machine in block 408 and transitions to a safe mode (in block 422). For instance, referring to the above, the first sub-state machines may include blocks 402, 404, and 406. Further, the control system may store states associated with the first sub-state machines blocks 402, 404, and 406 in the external system 20. The last-saved state may be a unique state ID associated with the “line priming” sub-state machine of block 406. When an interruption 420 (e.g., an event) occurs, the control system provides instructions to direct the bioprocessing system 10 to enter into a safe mode (e.g., block 422).

Once receiving an instruction to resume (e.g., from user selection in block 424), the control system may resume the manufacturing process in multiple phases. As indicated in block 426, a recovery initialization may take place first. The recovery initialization refers to an initial phase that may involve several recovery processes to function as a preliminary resumption process. Upon completion of the initial phase, the control system may cause display of a downtime popup (in block 428) in a user interface, and proceed to restart routing according to a recovery strategy (in block 430). As such, blocks 308, 310, and 312 of FIG. 3 may refer to blocks 424, 426, 428, and/or block 430, and the control system may perform a recovery strategy and resume the manufacturing of cells.

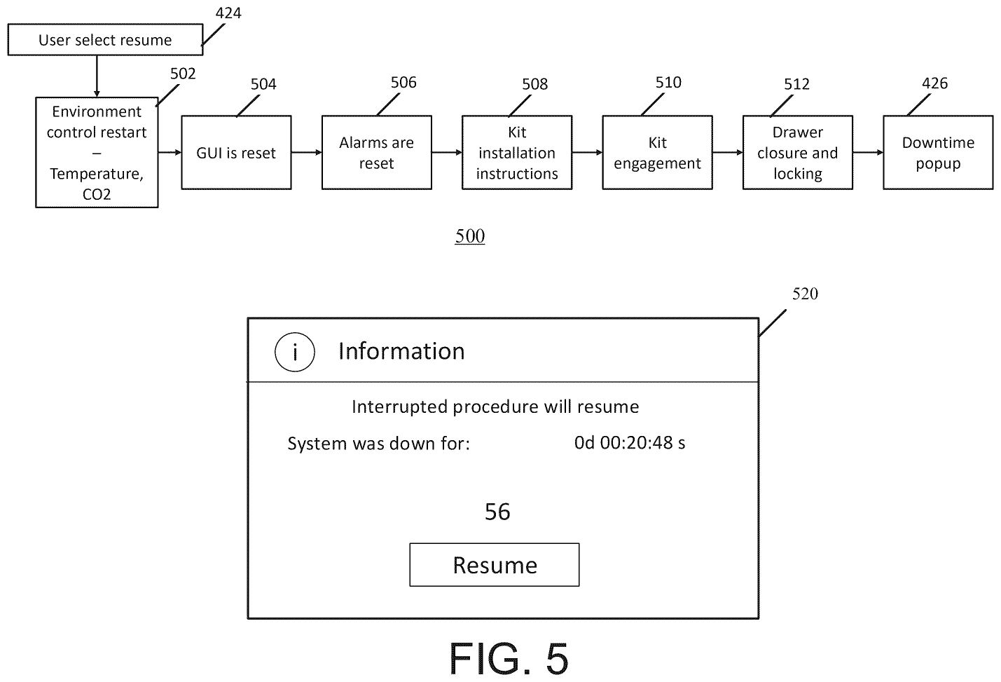

FIG. 5 is a simplified block diagram depicting exemplary recovery processes taken place after the user selects the resumption (in block 424) and prior to the display of an exemplary downtime popup (in block 428).

As shown in FIG. 5, after receiving the user input (in block 424), the control system may determine a series of processes 500 (e.g., corresponding to a sequence of sub-state machines and/or a series of states within a sub-state machine) for the bioprocessing system 10 (or the bioprocessing system 30) to carry out, including restart of environment control (in block 502), reset of graphic user interface (GUI) (in block 504), reset of alarms (in block 506), reset of kit (in blocks 508 and 510), drawer closure and locking (in block 512). Certain processes, such as the processes in block 502, 504, 506, 510, or 512, may be automated. In some variations, the control system may prompt user involvement for one or more processes. For example, in block 508, the control system may present kit installation instructions, prompting the user to inspect or reconfigure the kit.

For instance, referring back to FIG. 3 and block 310 as well as block 312, after determining the sub-state machine that was most recently executed, the control system may resume the manufacturing of the cells. To resume this manufacturing of the cells, the control system may determine a series of processes (e.g., processes 500) to perform in order to safely restart the manufacturing process (e.g., start the manufacturing process of the cells without damaging, destroying and/or otherwise altering the cells within the bioprocessing system 10).

After completion of the recovery processes, the control system may execute block 428, presenting the exemplary downtime popup 520. As shown in FIG. 5, the downtime popup 520 may indicate that the resumption is in progress. Additionally, it may include information indicating the duration of the system being in a down state and/or the estimated countdown to the completion of the resumption.

At block 430, the control system may restart routing based on a recovery strategy. For example, the control system may perform the process 300 as illustrated in FIG. 3 to determine the most recently executed sub-state machine (e.g., corresponding to the process “liquid transfer” in block 408) before pausing the manufacturing process. Additionally, the control system may determine that blocks 402, 404, and 406 are mandatory before executing block 408. As such, the control system may restart the bioprocessing system 10 by resuming at block 402 (e.g., “Open path”). Likewise, if an interruption occurs during blocks 402, 404, or 406, the control system may restart the bioprocessing system 10 by resuming at block 402. In certain scenarios, if the interruption happens during blocks 410 or 412, the control system may restart the bioprocessing system 10 at block 410 (“Stop pump”) or block 412 (“Close path”), respectively.

In essence, for certain application crash points (e.g., points where an event occurs and the manufacturing process has to be paused), the execution of the application may resume from the last executed state (e.g., the “Stop pump” state/block 410 from FIG. 4). In some scenarios, the execution of the application may resume from a previous executed state (e.g., the “Open path” state/block 402 from FIG. 4). For example, the control system may perform specific processes to resume the application from another “recovery point” according to predefined rules. For example, when the control system determines that the crash point is a particular state and/or within a particular sub-state machine, the control system may determine a target state within a target sub-state machine for the application to resume from. In some cases, before resuming the system (e.g., after the downtime popup in block 428 and before restarting the routing in block 430), the system may perform an addition recovery step. For example, if an interruption occurs during a state within a sub-state machine “perfusion” (e.g., with a protocol ID P762), an additional step involving cell settling is performed following the downtime popup (in block 428). This cell setting step may not be included in the sub-state machine P762. Nevertheless, this step is executed during the resumption process. The following examples of processes that may be performed by the control system are provided for illustrative purposes only and depict certain scenarios for ease of understanding.

Referring to FIG. 2, the application may be interrupted while executing the sub-state machine P701 (in block 204 “Kit test”). During the resumption, based on determining that the last executed sub-state machine is P701 (e.g., any state within this sub-state machine), the control system may resume the state machine 200 of the application to the initial state of the sub-state machine P701. As such, after the resumption, the application may restart the execution of the sub-state machine P701 from the beginning.

The application may be interrupted while executing the sub-state machine P702 (in block 222 “Volume reduction”). Depending on the interrupted state within this sub-state machine P702, the control system may determine a different recovery point for the application. For instance, if the interrupted state was related to a settling phase, such as when the bioprocessing system 10 was interrupted during settling time, the control system may resume the state machine 200 of the application from a recovery point prior to the interrupted state, so that the resumed application will redo the settling time.

The application may be interrupted while executing the sub-state machine P703 (in block 224 “Wash”). Similarly, if the interrupted state was one of a “settling” state, a “booster” state, or a state for “media and waste bags mass checks before starting wash” within the sub-state machine P703, the control system may resume the state machine 200 of the application from a recovery point prior to the interrupted state, allowing the resumed application to redo the interrupted state from the beginning. Alternatively and/or additionally, the control system may resume the interrupted sub-state machine based on user inputs. For example, if the interrupted state was a “perfusion” state, the control system may ask the user for optional settling period and continue with the remaining wash duration.

Similarly, when the application was interrupted while executing a sub-state machine of “Perfusion feeding,” the control system may ask the user for optional settling period and restart the feeding period upon resumption.

The application may be interrupted while executing a sub-state machine of “Bags installations.” For example, if the interrupted state was during bags installation instructions, the control system may determine to redo the instructions upon resumption.

In a sub-state machine of “Vessel mixing,” if the interrupted state was during constant vessel (CV) mixing, the control system may determine to redo the CV mixing upon resumption.

In a sub-state machine of “Cell bag mixing,” if the interrupted state was during mixing, the control system may provide instructions to ask the user to redo mixing upon resumption.

In a sub-state machine of P752 “Clear lines” (e.g., in block 212, 218, or 220), if the interrupted state was related to any point during line clearing, the control system may resume the application to perform line clearing from the beginning.

In a sub-state machine related to any of Vessel-to-bag, Bag-to-vessel, Bag-to-bag, or Vessel-to-vessel transfers (e.g., in block 216), if the interrupted state was during liquid transfer, the control system may resume the application to restart and transfer remaining mass.

In a sub-state machine of “Sampling,” if the interrupted state was during sampling processes, the control system may resume the application to restart sampling from the beginning.

In a sub-state machine of “Feeding,” if the interrupted state was during user prompts for bolus addition, the control system may resume the application to restart the sequence of user prompts. In essence, if an interruption happens during a user prompt, particularly in a sequence of user instructions, the control system may restart the application from the beginning of the sequence.

In a sub-state machine of “Small volume addition (syringe),” if the interrupted state was before filling, the control system may resume the application to restart the small volume addition (syringe) from the beginning.

In a sub-state machine of “Culture split,” if the interrupted state was during user prompts for split, the control system may resume the application to restart the sequence of user prompts.

FIG. 6 is an exemplary workflow to resume a state machine in accordance with one or more examples of the present disclosure. A control system controls the bioprocessing system 10/30 and the external system 20, as illustrated in FIGS. 1A-1C, to carry out the workflow 600. The exemplary blocks involved in the workflow 600 may be an exemplary implementation of the control system performing portions of the state machine 200, as depicted in FIG. 2, when encountering an event. The control system may perform the process 300 illustrated in FIG. 3 to carry out the workflow 600. The descriptions, illustrations, and processes of FIG. 6 are merely exemplary and the workflow 600 may use other descriptions, illustrations, and processes for resuming a bioprocessing system from an interruption event.

In this example, the control system is configured to execute a sequence of sub-state machines within the state machine 200 as depicted in FIG. 2: P301 202→P704 206→P750 208→P754 216. In the course of operation, the control system has stored, in the external system 20, a call stack with corresponding executed states as: state ID “1000” for the sub-state machine P301; state ID “2000” for the sub-state machine P704; state ID “3000” for the sub-state machine P750; state ID “1100” for the sub-state machine P754. Additionally, and/or alternatively, the control system has saved all variables associated with the executed sub-state machines corresponding to the call stack. In the case of crash, the variables' last values of all sub-state machines for a particular application (APP) are retrievable.

In the case of normal run, when the control system executes a sub-state machine within the state machine 200, the control system may use default values to configure the bioprocessing system 10 for initiating the sub-state machine. During the execution of the sub-state machine, the control system may retain variable values through callbacks for the sub-state machine.

In this example, at block 602, the control system loads a state machine associated with a selected application (e.g., with a root node 202 corresponding to a sub-state machine P301).

At block 604, the control system determines whether a previous run of the application was aborted.

Based on determining that the previous run was not aborted, at block 606, the control system loads default variable values corresponding to the sub-state machine P301. At block 608, the control system stores the first state corresponding to the sub-state machine P301 to the external system 20 in a suitable form. For example, the first state and/or the corresponding sub-state machine (P301) may be stored in extensible Markup Language (XML) code. The control system may include additional information in the saved XML code, such as variable values corresponding to the sub-state machine and/or the state. At block 610, the control system executes the state machine of the application starting with the first state corresponding to the sub-state machine P301.

At block 612, based on determining that the previous run was aborted, the control system prompts the user to select whether to resume the application. When the user selects to not resume the application, the control system may proceed to block 606 to restart the application.

When the user selects to resume the application, the control system proceeds to block 620 to resume the application according to a recovery strategy outlined in the blocks 620, 622, 624, 626, 628, and 630. In this example, a last stateID refers to a state ID that was last saved to a corresponding sub-state machine. For example, a last stateID may be saved to a completed state within a respective sub-state machine. Alternatively, a last stateID may be saved for an interrupted state within a respective sub-state machine after the occurrence of the interruption.

At block 620, the control system loads the last stateID for the sub-state machine P301 and the saved variable values.

At block 622, the control system determines whether the crashed state in P301 is a sub-state machine caller state. For example, the control system may determine, based on the last stateID for the sub-state machine P301, whether a subsequent sub-state machine within the state machine 200 was executed after P301. The control system may make the decision based on the last stateID for P301 and/or the saved variables.

If no, the control system directs the state machine to continue normal execution.

At block 624, based on determining that the crashed state is a sub-state machine caller state in P301, the control system calls the sub-state machine P704. Then, at block 626, the control system loads, from the external system 20, the last stateID (or other suitable state ID) and saved variable values associated with the sub-state machine P704.

At block 628, the control system determines whether the crashed state in P704 is a sub-state machine caller state. The control system may make the decision based on the last stateID for P704 and/or the saved variables. If no, the control system directs the state machine to continue normal execution. If yes, the control system may perform additional blocks similar to blocks 624 and 626. These blocks have been omitted from workflow 600 solely for brevity. When present and similar to blocks 626 and 626, the control system calls the next sub-state machine (e.g., P750) and loads, from the external system 20, the last stateID and saved variable values associated with the sub-state machine P750. The control system may then determine whether the crashed state in P750 is a sub-state machine caller state, and this may repeat until block 630.

At block 630, the control system determines whether the crashed state is a sub-state machine caller state in P754. The control system may repeat similar processes until the control system identifies the “last-level” sub-state machine associated with the crashed state. This way, the saved values, such as last stateIDs and/or saved variable values, are loaded until execution is directed to the last-level sub-state machine, where the interruption takes place. For example, based on the control system determining that the crashed state is associated with the sub-state machine 754, the control system guides the state machine to continue execution from the last-level sub-state machine as normal run (in block 610).

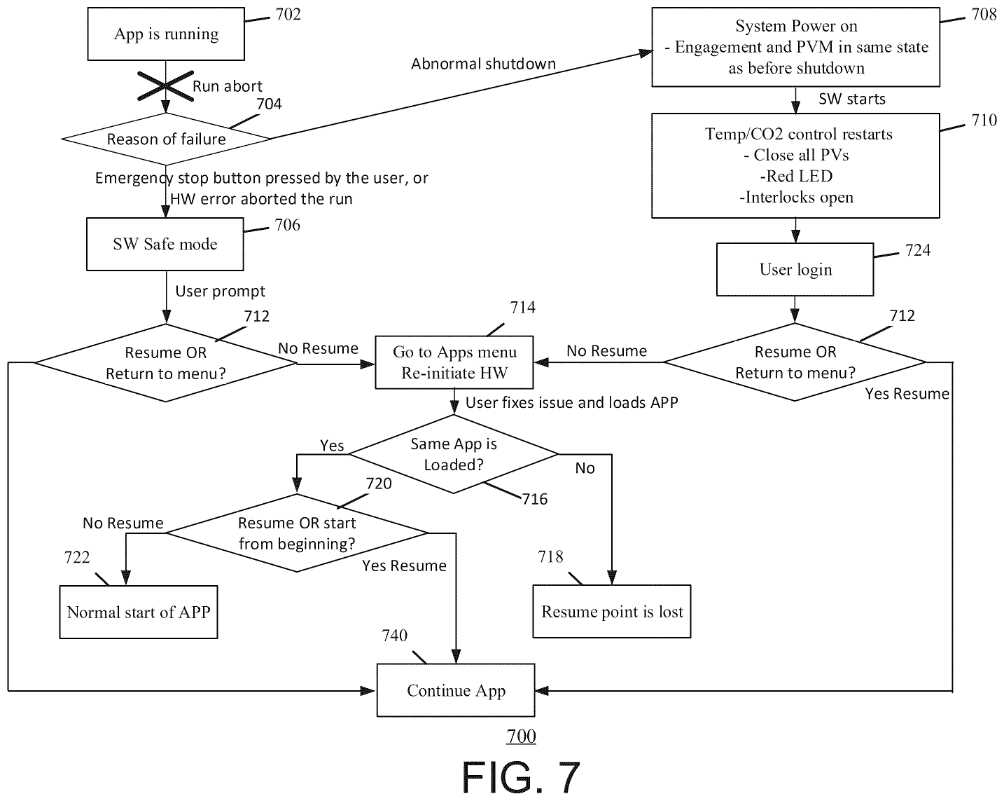

FIG. 7 is an exemplary workflow to resume a manufacturing process in accordance with one or more examples of the present disclosure. A control system controls the bioprocessing system 10/30 and the external system 20, as illustrated in FIGS. 1A-1C, to carry out the workflow 700. The exemplary procedures involved in the workflow 700 may be an exemplary implementation of the control system performing portions of a path in the state machine 200, as depicted in FIG. 2. The control system may perform the process 300 illustrated in FIG. 3 to carry out the workflow 700. The descriptions, illustrations, and processes of FIG. 7 are merely exemplary and the workflow 700 may use other descriptions, illustrations, and processes for resuming a bioprocessing system from an interruption event.

For example, the control system may perform the workflow 700 to determine whether to set the bioprocessing system 10 into a safe mode. At block 702, the control system runs an application (APP) to control a manufacturing process on the bioprocessing system 10. During the operation, the control system stores pertinent information in the external system 20, including state IDs (e.g., last-saved state IDs), a set of variables, and/or other suitable information corresponding to each sub-state machine in the application. The run was aborted.

At block 704, the control system may determine a reason of failure and subsequently decide on the appropriate course of action for the bioprocessing system 10 to pause. For example, the control system may identify that an emergency button was pressed by the user or a hardware (HW) error aborted the run. To this end, the bioprocessing system 10 may enter a safe mode (e.g., a software (SW) safe mode at block 706) after the interruption. In the safe mode, the bioprocessing system 10 may maintain certain conditions to preserve the samples in the system. For example, the bioprocessing system 10 may undertake operations such as maintaining temperature and carbon dioxide (CO2) levels within the system, closing all pinch valves (PVs), stopping pumps, restoring a tilted platform to a horizontal position, engaging disposable kit (DK), activating a red light (e.g., a red-light emitting diode (LED)), or opening interlocks.

Alternatively, the control system may undergo an abnormal shutdown (e.g., loss of power). When the power is restored, the engagement and pinch valves manifold (PVM) within the bioprocessing system 10 may be in the same state as before shutdown (at block 708). The control system may restore the software (SW) on the bioprocessing system 10, which may cause operations such as restarting the temperature or CO2 control, closing all PVs, activating a red light, or opening interlocks (at block 710).

Subsequently, the control system may determine whether and how to resume the application based on user inputs. As explained below, the control system may offer users various available options at different stages to choose the appropriate course of action.

When the bioprocessing system 10 is in the SW safe mode, the control system may prompt the user to determine whether to resume the application or return to the menu (at block 712).

If the user selects to resume the application (APP), the control system may perform the process 300 as shown in FIG. 3 to resume the APP and then continue the APP (at block 740). In some instances, the control system may perform the resumption in multiple phases as depicted in FIGS. 4 and 5. By performing the resumption process, the control system may direct a corresponding state machine to continue the interrupted application as demonstrated in FIG. 6.

If the user selects not to resume at this point, the control system may go to the APP menu to re-initiate the hardware (at block 714). Re-initiation of the HW may involve operations such as stopping temperature control, disengaging DK, opening all PVs, or activating a green light (e.g., a green LED). The user may fix issues and instruct the control system to load the application.

At this point, the control system may determine whether the same application is loaded (at block 716). The resume point (e.g., the recovery point) may be lost (at block 718) if the control system determine that the loaded application is different from the aborted one. Otherwise, if the same application is loaded, the control system may prompt the user to choose between initiating a normal start of the application (indicating no resume) (at block 722) or resuming and continuing the application (at block 740).

Referring back to the abnormal shutdown from block 704, when the bioprocessing system 10 is restarted after an abnormal shutdown, after the user logs in (at block 724), the control system may perform block 712 and direct the user to go through the same decision path as discussed above (from block 714). Alternatively, if the user selects to resume the APP, the control system may perform the process 300 as shown in FIG. 3 to resume the APP and then continue the APP (at block 740).

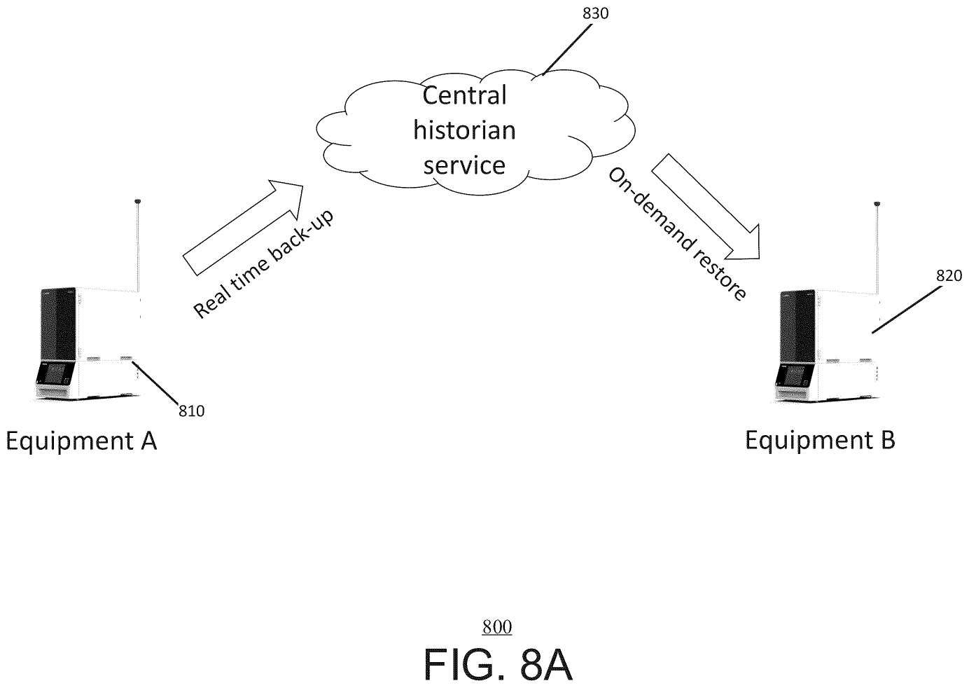

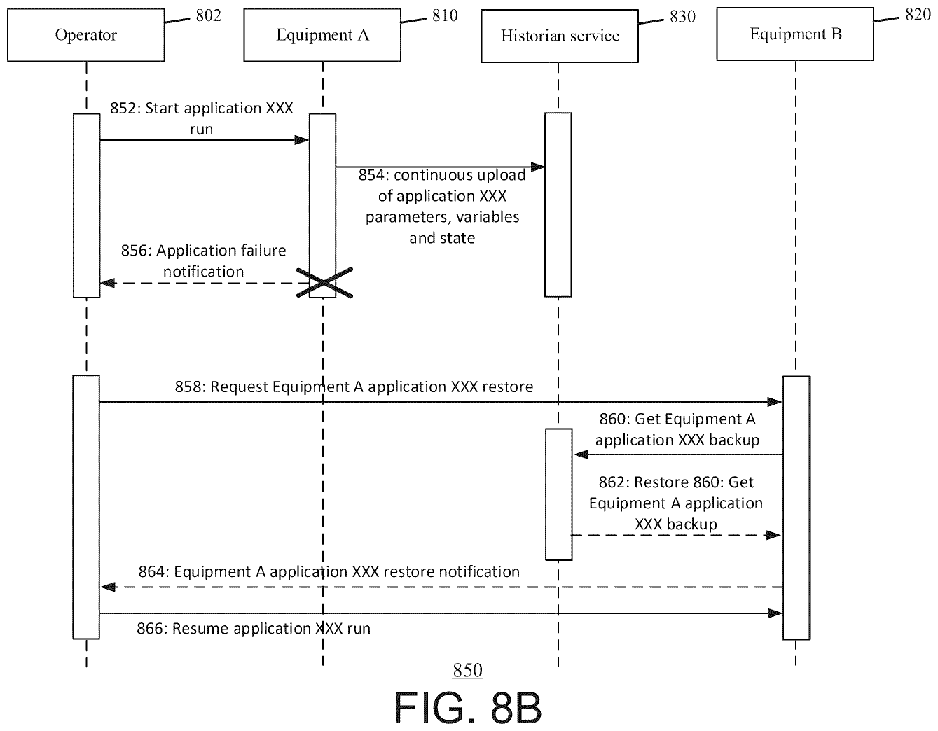

FIG. 8A depicts an exemplary environment in accordance with an example of the present disclosure. The entities within the environment 800 can take the form of any suitable systems described herein and are configured to execute the processes and workflows outlined herein.

As shown in FIG. 8A, the environment 800 includes a first bioprocessing system 810, a second bioprocessing system 820, and a central historian service 830 operating on a cloud platform. These components are interconnected through a network, facilitating communication between the first bioprocessing system 810 and the second bioprocessing system 820. Each bioprocessing system may be controlled by a corresponding control system within or connected to the respective system to control its operation.

In this example, the control system controls the first bioprocessing system 810 to transmit relevant data to the central historian service 830, facilitating the resumption of an application on a bioprocessing system using the stored data. For example, the first bioprocessing system 810 may perform real time back-up and/or on-demand back-up. In the event of resuming the application on the second bioprocessing system 820, the control system controls the second bioprocessing system 820 to execute on-demand restoration. For example, the control system retrieves the saved data from the central historian service 830 and configures the second bioprocessing system 820 to restore the process on the first bioprocessing system 810.

FIG. 8B is an exemplary workflow for resuming an application with reference to FIG. 8A. As shown in FIG. 8A, the application interrupted on equipment A 810 is resumed on equipment B 820 based on relevant data retrieved from the historian service 830. The workflow 850 may be performed by the control system of the equipment A and/or the control system of the equipment B. The user (e.g., operator 802) may instruct the control system(s) to execute the workflow 850 to resume the application on the equipment B.

At block 852, the operator 802 starts an application on the equipment A 810.

At block 854, the equipment A 810 continuously uploads, to the historian service 830, relevant data, such as parameters, variables, and states, associated with the application during normal operation. The stored data for the equipment A 810 may be referred to as the equipment A application backup.

At block 856, in an event of interruption, the equipment A 810 sends a notification to the operator 802 indicating the failure of the application.

At block 858, the operator 802 requests the equipment B 820 to restore the application on the equipment A 810.

At block 860, the equipment B 820 requests the equipment A application backup from the historian service 830.

At block 862, the equipment B 820 restores the equipment A application backup from the historian service 830.

At block 864, the equipment B sends a notification to the operator indicating the restoration of the equipment A application on the equipment B 820.

At block 866, the operator 802 instructs the equipment B 820 to resume the interrupted application.

Upon receiving the resumption instruction from the operator 802, the control system of the equipment B 820 may perform the process 300 and/or execute other suitable workflows disclosed herein to perform the resumption of the application on the equipment B 820.

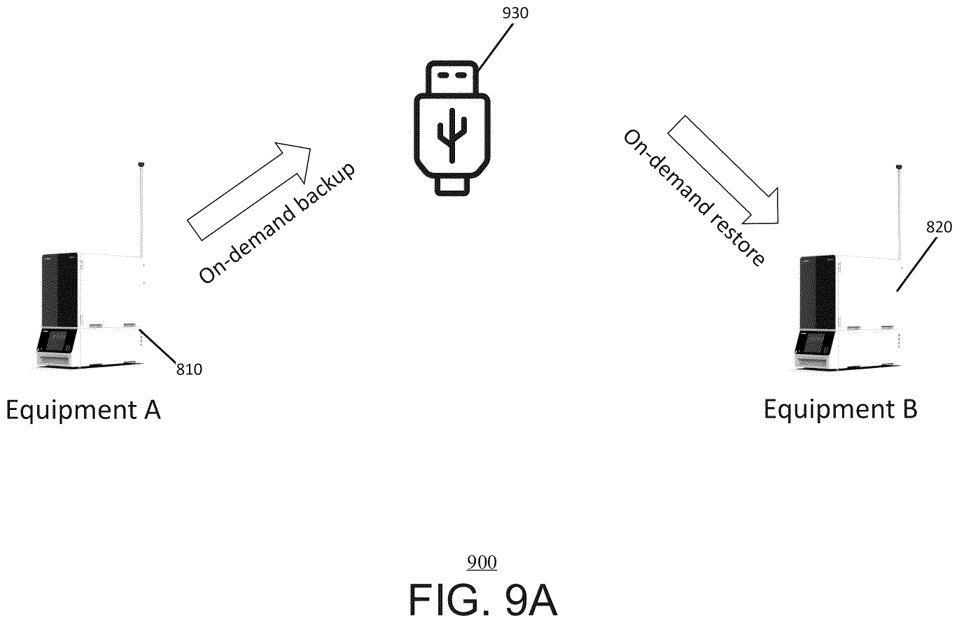

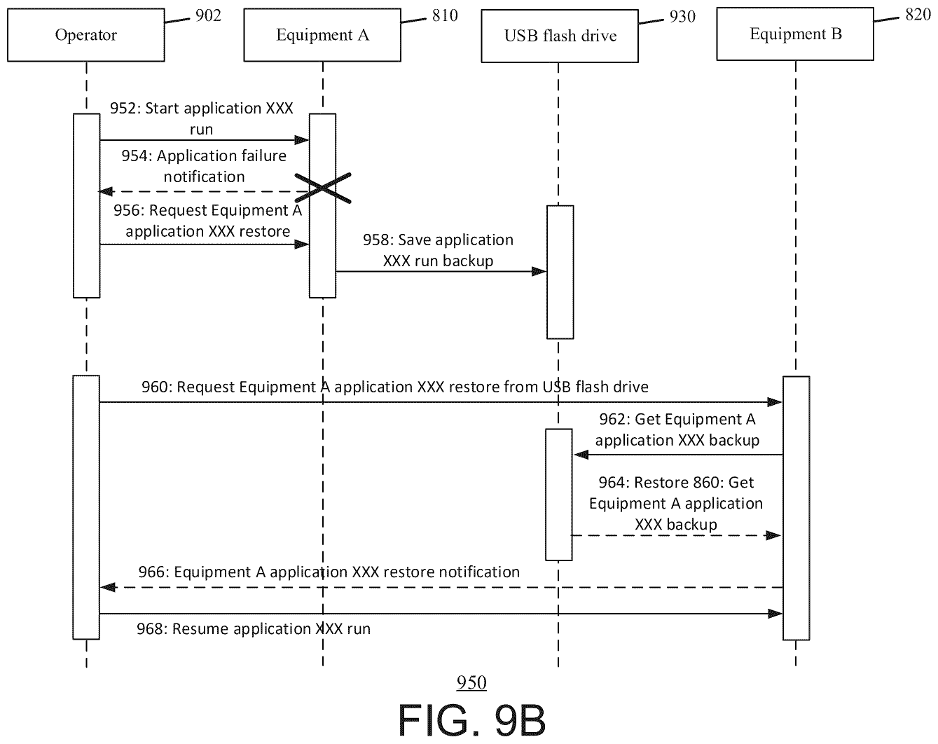

FIG. 9A depicts an exemplary environment in accordance with an example of the present disclosure. The entities within the environment 900 can take the form of any suitable systems described herein and are configured to execute the processes and workflows outlined herein.

Similar to the environment 800 as depicted in FIG. 8A, the environment 900 includes a first bioprocessing system 810 and a second bioprocessing system 820. Unlike the environment 800 shown in FIG. 8A, in the environment 900 of this example, an external storage device 930, such as a USB device, is utilized to restore necessary information, facilitating the backup of the application. Each bioprocessing system may be controlled by a corresponding control system within or connected to the respective system to control its operation.

The external storage device 930 is first connected to the first bioprocessing system 810. The first bioprocessing system 810 may perform real time back-up and/or on-demand back-up when connected to the external storage device 930.

After the interruption event, the user may decide to resume the interrupted application on the second bioprocessing system 820. To this end, the user may disconnect the external storage device 930 from the first bioprocessing system 810 and then connected it to the second bioprocessing system 820. To resume the application on the second bioprocessing system 820, the control system controls the second bioprocessing system 820 to execute on-demand restoration. For example, the control system retrieves the saved data from the external storage device 930 and configures the second bioprocessing system 820 to restore the process on the first bioprocessing system 810.

FIG. 9B is an exemplary workflow for resuming an application with reference to FIG. 9A. As shown in FIG. 9A, the application interrupted on equipment A 810 is resumed on equipment B 820 based on relevant data retrieved from the USB flash drive 930. The workflow 950 may be executed by the control system of the equipment A and/or the control system of the equipment B. The user (e.g., operator 902) may instruct the control system(s) to execute the workflow 950 to resume the application on the equipment B.

At block 952, the operator 902 starts an application on the equipment A 810. In some examples, the equipment A 810 may continuously store in the USB flash drive 930 relevant data, such as parameters, variables, and states, associated with the application during normal operation. The stored data for the equipment A 810 may be referred to as the equipment A application backup.

At block 954, in an event of interruption, the equipment A 810 sends a notification to the operator 902 indicating the failure of the application.

At block 956, the operator 902 requests the equipment A 810 to store a backup in the USB flash drive. The backup may be referred to as the equipment A application backup.

At block 958, the equipment A 810 stores the equipment A application backup in the USB flash drive 930.

At block 960, the operator 902 requests the equipment B 820 to restore the equipment A application backup.

At block 962, the equipment B 820 requests the equipment A application backup from the USB flash drive 930.

At block 964, the equipment B 820 restores the equipment A application backup from the USB flash drive 930.

At block 966, the equipment B 820 sends a notification to the operator 902 indicating the restoration of the equipment A application backup on the equipment B 820.

At block 968, the operator 902 instructs the equipment B 820 to resume the interrupted application.

Upon receiving the resumption instruction from the operator, the control system of the equipment B 820 may perform the process 300 and/or execute other suitable workflows disclosed herein to perform the resumption of the application on the equipment B 820.

A number of implementations have been described. d. Nevertheless, it will be understood that additional modifications may be made without departing from the scope of the inventive concepts described herein, and, accordingly, other examples are within the scope of the following claims. For example, it will be appreciated that the examples of the application described herein are merely exemplary. Variations of these examples may become apparent to those of ordinary skill in the art upon reading the foregoing description. The inventor expects skilled artisans to employ such variations as appropriate, and the inventor intends for the application to be practiced otherwise than as specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

It will further be appreciated by those of skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein as being performed by computing devices and/or components thereof may be carried out by according to processor-executable instructions and/or installed applications corresponding to software, firmware, and/or computer hardware.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the application and does not pose a limitation on the scope of the application unless otherwise claimed. No language in the specification should be construed as indicating any non-claimed element as essential to the practice of the application.

1. A method for resuming a bioprocessing system that is configured to manufacture cells for a cellular therapy, the method comprising:
executing, by one or more processors and using the bioprocessing system, one or more first sub-state machines, of a plurality of sub-state machines for manufacturing the cells, wherein each of the plurality of sub-state machines is associated with a node within a state machine, wherein the state machine comprises a decision tree with a plurality of branches and a plurality of nodes;
storing, by the one or more processors and in an external system, a plurality of states, wherein each of the plurality of states is saved into the external system based on completion of a state within a first sub-state machine, from the one or more first sub-state machine, for manufacturing the cells;
based on detecting an event, pausing, by the one or more processors, the manufacturing of the cells;
based on receiving user input indicating to resume the manufacturing of the cells, retrieving, by the one or more processors, a saved state from the external system, wherein the saved state is a state that was saved last to the external system;
comparing, by the one or more processors, the saved state with the state machine to determine a sub-state machine, from the one or more first sub-state machines, that was most recently executed by the bioprocessing system prior to pausing the manufacturing of the cells; and
resuming, by the one or more processors, the manufacturing of the cells based on the determined sub-state machine.

2. The method of claim 1, wherein each of the plurality of states stored in the external system is associated with a state identifier (ID), and wherein comparing the saved state with the state machine to determine the sub-state machine comprises:
comparing the state ID of the saved state with the state machine to determine a sub-state machine, from the plurality of sub-state machines, having a state with the same state ID as the saved state; and
determining the sub-state machine based on the comparison result.

3. The method of claim 1, further comprising:
storing, by the one or more processors and in the external system, a plurality of variables associated with operating conditions of the bioprocessing system, wherein a subset of the plurality of variables is saved into the external system based on completion of the sub-state machine, from the one or more first sub-state machines, for manufacturing the cells, and
wherein resuming the manufacturing of the cells is further based on using the subset of the plurality of variables associated with the determined sub-state machine.

4. The method of claim 3, wherein executing the sub-state machine, from the one or more first sub-state machines, begins with default values for the subset of the plurality of variables, and wherein the subset of the plurality of variables is periodically updated during the execution of the sub-state machine from the one or more first sub-state machines.

5. The method of claim 1, further comprising:
receiving a plurality of first user inputs indicating a plurality of parameters associated with manufacturing the cells; and
determining the one or more first sub-state machines based on the plurality of parameters.

6. The method of claim 1, wherein pausing the manufacturing of the cells based on detecting the event comprises:
providing one or more first instructions to one or more devices within the bioprocessing system to initiate a safe mode, and
wherein resuming the manufacturing of the cells comprises:
providing, based on the determined sub-state machine, one or more second instructions to the one or more devices within the bioprocessing system to stop the safe mode.

7. The method of claim 6, wherein the bioprocessing system maintains certain conditions to preserve the cells within the bioprocessing system in the safe mode, and
wherein providing the one or more first instructions to the one or more devices within the bioprocessing system to initiate the safe mode comprises one or more of:
maintaining temperature and carbon dioxide (CO2) levels within the bioprocessing system;
closing one or more pinch valves (PVs);
stopping one or more pumps;
restoring a tilted platform within the bioprocessing system to a horizontal position;
engaging a disposable kit (DK);
activating a red light; or
opening one or more interlocks.

8. The method of claim 1, wherein resuming the manufacturing of the cells comprises:
determining a plurality of recovery processes based on the determined sub-state machine;
performing the plurality of recovery processes; and
after performing the plurality of recovery processes, executing a subsequent sub-state machine, from the plurality of sub-state machines, to resume the manufacturing of the cells.

9. The method of claim 1, wherein the external system is a removable storage device.

10. The method of claim 1, wherein the external system is a network attached storage (NAS), and wherein storing the plurality of states comprises providing, by the one or more processors and to the NAS, the plurality of states via a network.

11. The method of claim 1, wherein resuming the manufacturing of the cells based on the determined sub-state machine comprises:
providing, to a second bioprocessing system, instructions to resume the manufacturing of the CAR T-cells based on the determined sub-state machine, wherein the second bioprocessing system is separate from the bioprocessing system that executed the one or more first sub-state machines.

12. The method of claim 1, wherein the external system comprises a database, and wherein the plurality of states are saved into the database in the external system based on a sequence of completing each sub-state machine of the plurality of sub-state machines.

13. The method of claim 1, wherein the cellular therapy is chimeric antigen receptor (CAR) T-cell therapy.

14. A non-transitory computer readable medium with instructions stored thereon for resuming a bioprocessing system that is configured to manufacture cells for a cellular therapy, wherein the instructions, when executed by one or more processors, causing the one or more processors to carry out:
executing, using the bioprocessing system, one or more first sub-state machines, of a plurality of sub-state machines for manufacturing the cells, wherein each of the plurality of sub-state machine is associated with a node within a state machine, wherein the state machine comprises a decision tree with a plurality of branches and a plurality of nodes;
storing, in an external system, a plurality of states, wherein each of the plurality of states is saved into the external system based on completion of a sub-state machine, from the one or more first sub-state machines, for manufacturing the cells;
based on detecting an event, pausing the manufacturing of the cells;
based on receiving user input indicating to resume the manufacturing of the cells, retrieving a saved state from the external system, wherein the saved state is a state that was saved last to the external system;
comparing the saved state with the state machine to determine a sub-state machine, from the one or more first sub-state machines, that was most recently executed by the bioprocessing system prior to pausing the manufacturing of the cells; and
resuming the manufacturing of the cells based on the determined sub-state machine.

15. The non-transitory computer readable medium of claim 14, wherein each of the plurality of states stored in the external system is associated with a state identifier (ID), and wherein comparing the saved state with the state machine to determine the sub-state machine comprises:
comparing an identifier associated with the saved state with the state machine to determine a sub-state machine, from the plurality of sub-state machines, having a state with the same state ID as the saved state; and
determining the sub-state machine based on the comparison result.

16. The non-transitory computer readable medium of claim 14, wherein the instructions, when executed by one or more processors, causing the one or more processors to further carry out:
storing, in the external system, a plurality of variables associated with operating conditions of the bioprocessing system, wherein a subset of the plurality of variables is saved into the external system based on completion of the sub-state machine, from the one or more first sub-state machines, for manufacturing the cells, and
wherein resuming the manufacturing of the cells is further based on using the subset of the plurality of variables associated with the determined sub-state machine.

17. The non-transitory computer readable medium of claim 16, wherein executing the sub-state machine begins with default values for the subset of the plurality of variables, and wherein the subset of the plurality of variables is periodically updated during the execution of the sub-state machine from the one or more first sub-state machines.

18. The non-transitory computer readable medium of claim 14, wherein the instructions, when executed by one or more processors, causing the one or more processors to further carry out:
receiving a plurality of first user inputs indicating a plurality of parameters associated with manufacturing the cells; and
determining the one or more first sub-state machines based on the plurality of parameters.

19. The non-transitory computer readable medium of claim 14, wherein pausing the manufacturing of the cells based on detecting the event comprises:
providing one or more first instructions to one or more devices within the bioprocessing system to initiate a safe mode, and wherein resuming the manufacturing of the cells comprises:
providing, based on the determined sub-state machine, one or more second instructions to the one or more devices within the bioprocessing system to stop the safe mode.

20. A system for resuming a bioprocessing system that is configured to manufacture cells for a cellular therapy, the system comprising:
a first device in communication with one or more processors; and
the one or more processors configured to:
perform, using the bioprocessing system, one or more first sub-state machines, of a plurality of sub-state machines for manufacturing the cells, wherein each of the plurality of sub-state machines is associated with a node from a state machine, wherein the state machine comprises a decision tree with a plurality of branches and a plurality of nodes;
store, in the first device, a plurality of states, wherein each of the plurality of states is saved into the first device based on completion of a sub-state machine, from the one or more first sub-state machines, for manufacturing the cells;
based on detecting an event, pause the manufacturing of the cells;
based on receiving user input indicating to resume the manufacturing of the cells, retrieving a saved state from the first device, wherein the saved state is a state that was saved last to the first device;
comparing the saved state with the state machine to determine a sub-state machine, from the one or more first sub-state machines, that was most recently executed by the bioprocessing system prior to pausing the manufacturing of the cells; and
resuming the manufacturing of the cells based on the determined sub-state machine.
